(19) United States
(12) Patent Application Publication
(10) Pub. No.: US 2004/0072824 A1
(43) Pub. Date: Apr. 15, 2004

(54) Methods and compositions for the treatment of cancer

(76) Inventors: Adam Telerman, Paris (FR); Robert Amson, Paris (FR); Marius Tuijnder, Mechelen (BE)

Correspondence Address: JONES DAY, 51 Louisiana Aveue, N.W, WASHINGTON, DC, 20001-2113, US

(21) Appl. No.: 10/385,509

(22) Filed: Mar. 12, 2003

Related U.S. Application Data
(63) Continuation-in-part of application No. 10/304,964, filed on Nov. 27, 2002.
Application No. 10/304,964 is a continuation of application No. 09/885,031, filed on Jun. 20, 2001, now abandoned.

(30) Foreign Application Priority Data
Jun. 1, 2001 (FR) 01/07285

(51) Int. Cl. A61K 31/5415; A61K 31/495; A61K 31/137

(52) U.S. Cl. 514/225.8; 514/255.04; 514/649

(57) ABSTRACT

Methods of treating and managing cancer are disclosed, which comprise the administration of an antihistaminic agents or structurally/functionally related to compound, optionally in combination with one or more additional anti-cancer agents. Pharmaceutical compositions, including single unit dosage forms, and kits useful in the treatment and management of cancer are disclosed. Also disclosed is a method of determining effectiveness of a cancer treatment.

This application is a continuation-in-part of U.S. application Ser. No. 10/304,964, filed Nov. 27, 2002, which is a continuation of U.S. application Ser. No. 09/885,031, filed Jun. 20, 2001, which claims priority to French Application No. 01/07285, filed Jun. 1, 2001, all of which are incorporated herein by reference. This application also claims priority to PCT/FR12/001861, filed Jun. 3, 2002, incorporated herein by reference.

### 1. FIELD OF THE INVENTION

The present invention relates to the use of various compounds including antihistaminic agents or structurally/functionally related compounds in the treatment and management of cancer.

### 2. BACKGROUND OF THE INVENTION

The incidence of cancer continues to climb as the general population ages, as new cancers develop, and as susceptible populations (e.g., people infected with AIDS) grow. A tremendous demand therefore exists for new methods and compositions that can be used to treat patients with cancer.

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multi-step process that begins with minor preneoplastic changes, which may under certain conditions progress to neoplasia.

The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3^{rd} ed., Mosby, St. Louis: 1993).

Although treatments for various cancers are known in the art, it is still difficult-if not impossible-to predict ab initio the effect a particular combination of drugs may have on a given form of cancer. Consequently, continued research is necessary to provide new cancer therapies that are safe and effective.

### 3. SUMMARY OF THE INVENTION

This invention is directed, in part, to the use of antihistaminic agents or structurally/functionally related compounds for the treatment and management of cancer. Thus, a first embodiment of the invention encompasses a method of inhibiting the growth of a cancer cell, which comprises contacting the cell with an antihistaminic agent or a structurally/functionally related compound, or a pharmaceutically acceptable salt, prodrug, solvate, hydrate or clathrate thereof. This method is particularly useful for the in vitro screening of the specificity and effectiveness of various antihistaminic agents or compounds that are structurally or functionally related to the antihistaminic agents.

Another embodiment encompasses a method of treating cancer, which comprises administering to a patient in need of such treatment a therapeutically effective amount of an antihistaminic agent or a structurally/functionally related compound, or a pharmaceutically acceptable salt, prodrug, solvate, hydrate or clathrate thereof.

Another embodiment of the invention encompasses a method of managing cancer, which comprises administering to a patient in need of such treatment a therapeutically effective amount of an antihistaminic agent or a structurally/functionally related compound, or a pharmaceutically acceptable salt, prodrug, solvate, hydrate or clathrate thereof.

Cancers that can be treated or managed according to methods of the invention include blood borne and solid tumors, as well as primary and metastatic cancers. Examples of cancers include, but are not limited to, leukemia, lymphoma, and cancer of the liver, lung, pancreas, stomach, thyroid, larygopharnx, skin, uterus, breast, colon, cervix, ovary, testis, prostate and rectum. Specific cancers are leukemia, melanoma, and cancer of breast, colon and lung.

A wide range of antihistaminic agents or structurally/functionally related compounds can be used in the methods of the invention. Specific examples of the compounds include, but not limited to, perphenazine, sertralin, thioridazine, chlorpromazine, paroxetine, flupentixol, fluphenazine, hydroxyzine, promethazine, and pharmaceutically acceptable salts, prodrugs, solvates, hydrates and clathrates thereof.

It is possible to use the compounds of the present invention in combination with one or more of other anti-cancer agents. Accordingly, another embodiment of the invention encompasses a method of treating or managing cancer comprising administering to a patient in need of such treatment or management a therapeutically effective amount of an antihistaminic agent or a structurally/functionally related compound, or a pharmaceutically acceptable salt, prodrug, solvate, hydrate or clathrate thereof, and a therapeutically effective amount of a second anti-cancer agent.

In a particular method encompassed by this embodiment, the cancer is leukemia. Examples of second anti-cancer agents that can be used in this method include, but are not limited to, prednisone, vincristine, anthracycline, asparaginase, cytarabine, etoposide, cyclophosphamide, methotrexate, leucovorin, cytosine arabinose, corticosteroids, daunorubicin, idarubicin, 6-thioguanine, chlorambucil, fludarabine, interferon α, deoxyconformycin, 2-chlorodeoxyadenosine, hydroxyurea, 6-myelopurine, 6-thioguanine, melphalan, and pharmaceutically acceptable salts, prodrugs, solvates, hydrates and clathrates thereof.

In another particular method, the cancer is melanoma. Examples of second anti-cancer agents that can be used in this method include, but are not limited to, dacarbazine, nitrosoureas carmustine, lomustine, cisplatin, interleukin-2, and pharmaceutically acceptable salts, prodrugs, solvates, hydrates and clathrates thereof.

In another particular method, the cancer is breast cancer. Examples of second anti-cancer agents that can be used in this method include, but are not limited to, cyclophosphamide, 5-fluorouracil, methotrexate, doxorubicin, tamoxifen, progestins, aromatase inhibitors, aminoglutethimide, letrozole, paclitaxel, docetaxel, navelbine, capecitabine, mitomycin C, prednisone, taxane, vinblastine, and pharmaceutically acceptable salts, prodrugs, solvates, hydrates and clathrates thereof.

In another particular method, the cancer is colon cancer. Examples of second anti-cancer agents that can be used in this method include, but are not limited to, fluorouracil, folinic acid, levamisole, leucovorin, oxaliplatin, irinotecan, and pharmaceutically acceptable salts, prodrugs, solvates, hydrates and clathrates thereof.

In another particular method, the cancer is lung cancer. Examples of second anticancer agents that can be used in this method include, but are not limited to, cisplastin, topoisomerase inhibitors, carboplatin, vinorelbine, vincristine, vinblastine, docetaxel, oxaliplatin, paclitaxel, and pharmaceutically acceptable salts, prodrugs, solvates, hydrates and clathrates thereof.

Another embodiment of the invention encompasses pharmaceutical compositions and single unit dosage forms that can be used for the treatment or management of cancer, which comprise an antihistaminic agent or a structurally/functionally related compound, or a pharmaceutically acceptable salt, prodrug, solvate, hydrate or clathrate thereof, and a second anti-cancer agent. Pharmaceutical dosage forms include those suitable for oral, mucosal, parenteral, sublingual, transdermal, buccal or topical administration to a patient. Specific dosage forms are suitable for parenteral or oral administration.

Another embodiment of the invention encompasses a kit that can be used for the treatment or management of cancer, which comprises a single unit dosage form of an antihistaminic agent or a structurally/functionally related compound, or a pharmaceutically acceptable salt, prodrug, solvate, hydrate or clathrate thereof, and a single unit dosage form of a second anti-cancer agent.

Another embodiment of the invention encompasses a method of determining whether a cancer patient will respond to a cancer treatment comprising testing for overexpression of TPT1 gene in cancer cells from the patient, wherein the cancer treatment is administration of an antihistaminic agent or a structurally/functionally related compound, or a pharmaceutically acceptable salt, prodrug, solvate, hydrate or clathrate thereof.

Another embodiment of this invention encompasses a method of determining the effectiveness of a cancer treatment comprising: obtaining cancer cells from a patient being treated at a first time and at a second time; and determining if expression of TPT1 gene in cells obtained at the second time is less than the expression of TPT1 gene in cells obtained at the first time; wherein the first time is earlier than the second time.

### 3.1. BRIEF DESCRIPTION OF FIGURES

Certain aspects of the invention can be understood with reference to the figures described below:

FIG. 1 illustrates the effect of hydroxyzine dihydrochloride (“Atranine A”, Atarax®), brompheniramine maleate (Dimegan®), promethazine (“Atranine C”, Phenergan®) and dexchlorpheniramine maleate (Polaramine®) in myeloid leukemia K562 cells;

FIG. 2 illustrates the effect of hydroxyzine, brompheniramine maleate, promethazine and dexchlorpheniramine maleate in premonocytic leukemia U937 cells;

FIG. 3 illustrates the effect of hydroxyzine, brompheniramine maleate, promethazine and dexchlorpheniramine maleate in acute leukemia T cells derived from Jurkat cell line;

FIG. 4 illustrates the effect of hydroxyzine, brompheniramine maleate, promethazine and dexchlorpheniramine maleate in breast ductal carconima cells derived from T47-D cell line;

FIG. 5 illustrates the effect of hydroxyzine, brompheniramine maleate, promethazine and dexchlorpheniramine maleate in breast ductal carcinoma cells derived from MCF7 cell line;

FIG. 6 illustrates the effect of hydroxyzine, brompheniramine maleate, promethazine and dexchlorpheniramine maleate in mammary gland carcinoma cells derived from BT20 cell line;

FIG. 7 illustrates the effect of hydroxyzine, brompheniramine maleate, promethazine and dexchlorpheniramine maleate in immortalized, non-tumorigenic breast epithelium cells derived from 184B5 cell line;

FIGS. 8 and 9 illustrate the effect of hydroxyzine and promethazine in lymphocytes from healthy donors;

FIG. 10A illustrates that the growth of colorectal adenocarcinoma cells derived from LoVo cell line are unaffected by hydroxyzine and promethazine;

FIG. 10B illustrates the effect of hydroxyzine and promethazine in immortalized, non-tumorigenic breast luminal epithelium cells derived from 184B5 cell line;

FIG. 11 illustrates the antineoplastic activity of promethazine (Phenergan®) in U 937 cells injected in scid/scid mice;

FIGS. 12-14 illustrates the effect of the compounds of the present invention in inhibiting the growth of various cancer cell lines;

FIG. 15 illustrates the effect of the compounds of the present invention in inhibiting the growth of U937 cells;

FIG. 16 illustrates the effect of the compounds of the present invention at a concentration of 10⁻⁵M in inhibiting the viability of fresh leukemic cells ex vivo;

FIG. 17 illustrates the effect of the compounds of the present invention at a concentration of 10⁻⁶M in inhibiting the viability of fresh leukemic cells ex vivo;

FIG. 18 illustrates the effect of the compounds of the present invention in reducing the cell viability in various cancer cell lines;

FIG. 19 illustrates the curative effects of S60 and S59 in palpable tumors in U937 cells injected in scid/scid mice;

FIG. 20 illustrates the curative effects of S64, hydroxyzine (Atarax®), promethazine, A37 and SQ42 in MDA-MB-231 cells injected in scid/scid mice;

FIG. 21 illustrates the preventive effects of S60, S59 and promethazine in tumor development in U937 cells injected in scid/scid mice;

FIG. 22 illustrates the up-regulation of TCTP expression in tissues from various cancerous organs and their respective normal counterparts;

FIG. 23 illustrates the expression of TCTP following the treatment by various compounds of the present invention; and

FIG. 24 illustrates the induction of caspase 3 and 7 activities by the compounds of the present invention in U937 cell line.

### 3.2. DEFINITIONS

As used herein and unless otherwise indicated, the term “antihistaminic agent” encompasses antagonists of histamine receptors.

As used herein, the term “structurally related compound” encompasses compounds that possess structures similar to those of antihistaminic agents. Examples include, but are not limited to, derivatives, salts, metabolites, prodrugs, hydrates, solvates, or optical isomers of antihistaminic agents.

The term “functionally related compound”, as used herein, encompasses compounds that inhibit the expression of the TPT1 gene in humans, and inhibit or block the function of the protein encoded by the TPT1 gene. More broadly, the term also encompasses compounds that inhibit or block the formation or function of products resulting from a metabolic chain controlled directly or indirectly by the expression of TPT1 gene.

As used herein, the term “inhibit” means induction of reduction in functional capacity or level of formation of a given molecule. As used herein, the phrase that a compound that “inhibits” the function or expression of a molecule means that the compound causes about 10 percent or more, specifically 30 percent or more, more specifically 60 percent or more, and most specifically 100 percent in reduction of functional capacity or level of product formation of the molecule.

As used herein and unless otherwise indicated, the term “pharmaceutically acceptable salt(s)” refers to a non-toxic acid or base addition salt. In particular, basic chemical moieties are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions. Suitable organic acids include, but are not limited to, maleic, fumaric, benzoic, ascorbic, succinic, acetic, formic, oxalic, propionic, tartaric, salicylic, citric, gluconic, lactic, mandelic, cinnamic, oleic, tannic, aspartic, stearic, palmitic, glycolic, glutamic, gluconic, glucaronic, saccharic, isonicotinic, methanesulfonic, ethanesulfonic, p-toluenesulfonic, benzenesulfonic acids, or pamoic (i.e., 1,1′-methylene-bis-(2-hydroxy-3-naphthoate) acids. Suitable inorganic acids include, but are not limited to, hydrochloric, hydrobromic, hydroiodic, sulfuric, phosphoric, or nitric acids. Compounds that include an amine moiety can form pharmaceutically acceptable salts with various amino acids, in addition to the acids mentioned above.

Chemical moieties that are acidic in nature are capable of forming base salts with various pharmacologically acceptable cations. Examples of such salts are alkali metal or alkaline earth metal salts and, particularly, calcium, magnesium, sodium, lithium, zinc, potassium, or iron salts.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of the antihistaminic agents of the present invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of the compounds of the present invention that include —NO, —NO₂, —ONO, or —ONO₂ moieties.

As used herein to describe a compound or chemical moiety, the term “derivative” means a compound or chemical moiety wherein the degree of saturation of at least one bond has been changed (e.g., a single bond has been changed to a double or triple bond) or wherein at least one hydrogen atom is replaced with a different atom or a chemical moiety. Examples of different atoms and chemical moieties include, but are not limited to, halogen, oxygen, nitrogen, sulfur, hydroxy, methoxy, alkyl, amine, amide, ketone, and aldehyde.

As used herein and unless otherwise indicated, the terms “biohydrolyzable carbamate,” “biohydrolyzable carbonate,” “biohydrolyzable ureide,” “biohydrolyzable phosphate” mean a carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein and unless otherwise indicated, the term “biohydrolyzable ester” means an ester of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, alkoxyacyloxy esters, alkyl acylamino alkyl esters, and choline esters.

As used herein and unless otherwise indicated, the term “biohydrolyzable amide” means an amide of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable amides include, but are not limited to, lower alkyl amides, amino acid amides, alkoxyacyl amides, and alkylaminoalkylcarbonyl amides.

### 4. DETAILED DESCRIPTION OF THE INVENTION

The present invention is based, in part, on the discovery that certain antihistaminic agents and compounds of related structures or functions exhibit strong and selective anticancer activity against various tumors. In particular, Applicants discovered that during tumor reversion, certain genes are overexpressed during the tumor phase in comparison with the reversion phase. One of these overexpressed genes is translationally controlled tumor protein, or TPT1, which encodes histamine releasing factor. For example, Applicants discovered this phenomenon in the tumor cell line U937, which is capable of reverting to provide what is termed a US cell, which no longer exhibits malignant phenotype characteristics associated with the original tumor cells. In this particular example, Applicants found that 248 TPT1 were present out of about 2,000 sequences in the U937 cell line, whereas only 2 TPT1 clones were identified in the US cell line. This research catalyzed Applicants' discovery of the importance of the histamine activation pathway in the phenomenon of tumor reversion.

The present invention relates to the use of compounds that inhibit or prevent the expression of the TPT1 gene, inhibit or block the function of the protein encoded by TPT1 gene. Moreover, the invention also encompasses the use of compounds that inhibit or prevent the formation of products controlled by the TPT1 gene, e.g., products resulting from a metabolic chain controlled directly or indirectly by the expression of TPT1 gene. One such product is histamine.

### 4.1. Methods of Treatment and Management

This invention encompasses methods of treating cancer, which comprise administering to a patient (e.g., a human) in need of such treatment a therapeutically effective amount of an antihistaminic agent or a structurally/functionally related compound, or a pharmaceutically acceptable salt, prodrug, hydrate, solvate or clathrate thereof.

The invention also encompasses a method of managing cancer, which comprises administering to a patient in need of such management a prophylactically effective amount of an antihistaminic agent or a structurally/functionally related compound, or a pharmaceutically acceptable salt, prodrug, hydrate, solvate or clathrate thereof. As used herein and unless otherwise indicated, the term “managing” encompasses preventing the recurrence of cancer in a patient who had suffered from cancer, lengthening the time a patient who had suffered from cancer remains in remission, preventing the occurrence of cancer in patients at risk of suffering from cancer (e.g., patients who had been exposed to high amounts of radiation or carcinogenic materials, such as asbestos; patients infected with viruses associated with the occurrence of cancer, such as, but not limited to, HIV and Kaposi's sarcoma-associated herpesvirus; and patients with genetic predispositions to cancer, such as those suffering from Downs syndrome), and preventing the occurrence of malignant cancer in patients suffering from pre-malignant or non-malignant cancers.

As used herein, the phrases “therapeutically effective amount” and “prophylactically effective amount” refer to an amount that provides a therapeutic benefit in the treatment, prevention, or management of cancer. The specific amount that is therapeutically effective can be readily determined by ordinary medical practitioner, and may vary depending on factors know in the art, such as the type of cancer, the patient's history and age, the stage of cancer, the administration of other anti-cancer agents, including radiation therapy.

Methods of the invention can be used to treat and manage patients suffering from primary and metastatic cancer. They further encompass methods of treating patients who have been previously treated for cancer, as well as those who have not previously been treated for cancer. The invention encompasses first-line, second-line, third-line and further lines cancer treatments.

Cancers that can be treated and managed using methods of the invention include but are not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Additional examples of specific cancers include, but are not limited to: AIDS associated leukemia and adult T-cell leukemia lymphoma; anal carcinoma; astrocytoma; biliary tract cancer; cancer of the bladder, including bladder carcinoma; brain cancer, including glioblastomas and medulloblastomas; breast cancer, including breast carcinoma; cervical cancer; choriocarcinoma; colon cancer including colorectal carcinoma; endometrial cancer; esophageal cancer; Ewing's sarcoma; gastric cancer; gestational trophoblastic carcinoma; glioma; hairy cell leukemia; head and neck carcinoma; hematological neoplasms, including acute and chronic lymphocytic and myelogeneous leukemia; hepatocellular carcinoma; Kaposi's sarcoma; kidney cancer; multiple myeloma; intraepithelial neoplasms, including Bowen's disease and Paget's disease; liver cancer; lung cancer including small cell carcinoma; lymphomas, including Hodgkin's disease, lymphocytic lymphomas, non-Hodgkin's lymphoma, Burkitt's lymphoma, diffuse large cell lymphoma, follicular mixed lymphoma, and lymphoblastic lymphoma; lymphocytic leukemia; neuroblastomas; oral cancer, including squamous cell carcinoma; ovarian cancer, including those arising from epithelial cells, stromal cells, germ cells and mesenchymal cells; pancreatic cancer; prostate cancer; rectal cancer; sarcomas, including soft tissue sarcomas, leiomyosarcoma, rhabdomyosarcoma, liposcarcoma, fibrosarcoma, and osteosarcoma; skin cancer, including melanoma, Kaposi's sarcoma, basal cell cancer and squamous cell cancer; testicular cancer, including testicular carcinoma and germinal tumors (e.g., semicoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilm's tumor.

In a specific method, the compounds of the present invention are used to inhibit the growth of a cancer cell, wherein the cancer is leukemia, lymphoma, melanoma, or cancer of the liver, lung, pancreas, stomach, thyroid, larygopharnx, skin, uterus, breast, colon, cervix, ovary, testis, prostate or rectum. More specifically, the compounds of the present invention are effective in inhibiting the growth of a cancer cell, wherein the cancer is leukemia, melanoma, or cancer of breast, colon or lung.

The compounds used in methods and compositions of the invention include a wide variety of compounds, the suitability of which can be readily determined by those of ordinary skill using methods disclosed herein and known in the art. Typically, the compounds of this invention would encompass antihistaminic agents and compounds that are structurally or functionally related to the antihistaminic agents. Preferred compounds of the invention are antihistaminic agents.

Some examples of the antihistaminic agents that can be used for the purpose of this invention include, but are not limited to: alkylamines such as acrivastine, brompheniramine, chlorpheniramine and tripolidine; ethanolamine derivatives such as carbinoxamine, clemastine, diphenhydramine, dimenhydrinate and doxylamine; ethyleneamine derivatives such as mepyramine-type compounds, pyrilamine and tripelennamine; phenothiazine derivatives such as dimethiothiazine, hydroxyethylpromethazine, isothipendyl, mequitazine, methdilazine, oxomemazine, promethazine, propiomazine, thiazinamium and trimeprazine; piperidine derivatives such as astemizole, fexofenadine, levocabastine, loratadine, tefenadine and mizolastine; piperazine derivatives such as buclizine, cetirizine, cyclizine, flumarizine, homochlorcycline, hydroxyzine, meclizine, niaprazine and oxatomide; homologues and analogues thereof; and pharmaceutically acceptable salts, prodrugs, hydrates, solvates and clathrates thereof.

Examples of other structurally and functionally related compounds include, but are not limited to: dopamine antagonists such as, but not limited to, chlorpromazine, thioxanthenes including thioridazine; selective serotonin uptake inhibitors such as, but not limited to, clomipramine, nefazodone, paroxetine, sertraline, fluoxetine, fluvoxamine and citalopram; antidepressants such as, but not limited to, amitryptiline, doxepin, nortryptiline, venlafaxine trazodone, nefazodone, mianserine and minalcitran; other compounds such as flupentixol, fluphenazine, clemastine, fumarate, pyrimethamine, maprotiline, perphenazine, cyproheptadine, ketotifen, imipramine, levomepromazine, promazine, chlorprothixene, haloperidol, chloroquine; and pharmaceutically acceptable salts, prodrugs, hydrates, solvates and clathrates thereof.

Specific compounds include, but are not limited to, hydroxyzine, promethazine, perphenazine, sertraline, thioridazine, chlorpromazine, paroxetine, flupentixol, fluphenazine, and pharmaceutically acceptable salts, prodrugs, hydrates, solvates and clathrates thereof. In certain embodiments of the invention, the antihistaminic agent is not polaramine.

This invention encompasses the use of the compounds of this invention in combination with one or more second anti-cancer agents, or pharmaceutically acceptable salts, prodrugs, hydrates, solvates or clathrates thereof. For example, the compounds of this invention can be administered simultaneously or sequentially with antineoplastic agents such as antimetabolites, alkylating agents, spindle poisons and/or intercalating agents, and proteins such as interferons.

Examples of particular second anti-cancer agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracycline; anthramycin; aromatase inhibitors; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; chlorodeoxyadenosine; cirolemycin; cisplatin; cladribine; corticosteroids; crisnatol mesylate; cyclophosphamide; cytarabine; cytosine arabinose; dacarbazine; dactinomycin; daunorubicin hydrochloride; decitabine; deoxyconformycin; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; folinic acid; fosquidone; fostriecin sodium; gemcitabine; gemcitabine hydrochloride; hydroxyurea; idarubicin hydrochloride; ifosfamide; ilmofosine; interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; leucovorin; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; myelopurine; navelbine; nitrosoureas camustine; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; progestins; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; taxane; tecogalan sodium; tegafur; teloxantrone hydrochloride; temoporfin; teniposide; teroxirone; testolactone; thiamiprine; thioguanine; thiotepa; tiazofurin; tirapazamine; topoisomerase inhibitors; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Still other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard second anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

The determination of the identity and amount of second anti-cancer agent(s) to use in a method of the invention can be readily made by ordinarily skilled medical practitioners using standard techniques known in the art, and will vary depending on the type and severity of cancer being treated.

For example, second anti-cancer agents that can be used to treat leukemia include, but are not limited to, prednisone, vincristine, anthracycline, asparaginase, cytarabine, etoposide, cyclophosphamide, methotrexate, leucovorin, cytosine arabinose, corticosteroids, daunorubicin, idarubicin, 6-thioguanine, chlorambucil, fludarabine, interferon α, deoxyconformycin, 2-chlorodeoxyadenosine, hydroxyurea, 6-myelopurine, 6-thioguanine and/or melpharan.

Examples of the second anti-cancer agents that can be used to treat melanoma include, but are not limited to, dacarbazine, nitrosoureas carmustine, lomustine, cisplatin and/or interleukin-2; examples of the second anti-cancer agents that can be used to treat breast cancer include, but are not limited to, cyclophosphamide, 5-fluorouracil, methotrexate, doxorubicin, tamoxifen, progestins, aromatase inhibitors, aminoglutethimide, letrozole, paclitaxel, docetaxel, navelbine, capecitabine, mitomycin C, prednisone, taxane and/or vinblastine.

Examples of the second anti-cancer agents that can be used to treat colon cancer include, but are not limited to, fluorouracil, folinic acid, levamisole, leucovorin, oxaliplatin or irinotecan.

Examples of the second anti-cancer agents that can be used to treat lung cancer include, but are not limited to, cisplastin, topoisomerase inhibitors, carboplatin, vinorelbine, vincristine, vinblastine, docetaxel, oxaliplatin or paclitaxel.

The compounds of this invention and second anti-cancer agents can be administered to patients simultaneously or sequentially by the same or different routes of administration. The suitability of a particular route of administration employed for a particular compound will depend on the compound itself (e.g., whether it can be administered orally without decomposing prior to entering the blood stream) and the disease being treated. For example, treatment of tumors on the skin or on exposed mucosal tissue may be more effective if one or both active ingredients are administered topically, transdermally or mucosally (e.g., by nasal, sublingual, buccal, rectal, or vaginal administration). Treatment of tumors within the body, or prevention of cancers that may spread from one part of the body to another, may be more effective if one or both of the active ingredients are administered parenterally or orally. Similarly, parenteral administration may be preferred for the acute treatment of a disease, whereas transdermal or subcutaneous routes of administration may be employed for chronic treatment or prevention of a disease. Preferred routes of administration for the anti-cancer agents are known to those of ordinary skill in the art.

### 4.2. Pharmaceutical Compositions

This invention encompasses pharmaceutical compositions comprising an antihistaminic agent or a structurally/functionally related compound, or a pharmaceutically acceptable salt, prodrug, hydrate, solvate or clathrate thereof, and a second anti-cancer agent, or a pharmaceutically acceptable salt, prodrug, hydrate, solvate or clathrate thereof. The compounds of this invention and second anti-cancer agents that can be incorporated into such compositions are disclosed herein (e.g., in Section 4.1., above).

Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

4.2.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

4.2.2. Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.2.3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients (i.e., the compounds of this invention and second anti-cancer agents) disclosed herein can also be incorporated into the parenteral dosage forms of the invention.

4.2.4. Transdermal, Topical and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington 's Pharmaceutical Sciences, 16^{th} and 18^{th} eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.2.5. Kits

In some cases, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a single unit dosage form of an antihistaminic agent or a structurally/functionally related compound, or a pharmaceutically acceptable salt, solvate, hydrate, prodrug, or clathrate thereof, and a single unit dosage form of a second anti-cancer agent, or a pharmaceutically acceptable salt, solvate, hydrate, prodrug, or clathrate thereof. Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 4.3 DETERMINATION OF THE EFFECTIVENESS OF CANCER TREATMENT

As discussed herein above, Applicants have discovered that overexpression of TPT1 gene is observed in cancer cells, but not in normal cells. Certain compounds of the present invention are found to be effective in suppressing the expression of TPT1 gene. Based on this, it is possible to determine whether a cancer patient will be responsive to the administration of the compounds of the present invention by testing the overexpression of TPT1 gene. Accordingly, this invention encompasses a method of determining whether a cancer patient will respond to a cancer treatment comprising testing for overexpression of TPT1 gene in cancer cells from the patient, wherein the cancer treatment is administration of an antihistaminic agent or a structurally/functionally related compound, or a pharmaceutically acceptable salt, prodrug, solvate, hydrate or clathrate thereof.

The overexpression of TPT1 gene can be monitored using techniques well known in the art. One such technique is to monitor the cellular level of the product of TPT1 gene, namely TCTP, based on electrophoresis methods. Typically, after visualization, the intensity of the band corresponding to TCTP can be quantified using various techniques including, but not limited to, densitometry, fluorometry, spectrometry and luminometry.

Using the quantification, it is possible to determine whether TCTP is present at a higher level in cancer cells than in normal cells. As used herein, the term “overexpression” means that a higher level of TCTP is present in cancer cells as compared with normal cells. As used herein, “overexpression” would result in about 20 percent or more, specifically about 50 percent or more, more specifically about 100 percent or more, and most specifically about 200 percent or more TCTP formation in cancer cells as compared with normal cells.

Likewise, it is also possible to determine whether a cancer treatment is effective in a patient receiving the treatment by monitoring the decreased expression of TPT1 gene as the treatment progresses. Accordingly, another embodiment of this invention is directed to a method of determining the effectiveness of a cancer treatment comprising: obtaining cancer cells from a patient being treated at a first time and at a second time; and determining if expression of TPT1 gene in cells obtained at the second time is less than the expression of TPT1 gene in cells obtained at the first time; wherein the first time is earlier than the second time.

### 5. EXAMPLES

Particular aspects and advantages of the present invention may be further demonstrated by the following non-limiting examples.

### 5.1. Example 1

### Specific Cytotoxicity of the Compounds of the Invention

The cytotoxic activity of the following four compounds were tested: hydroxyzine dihydrochloride (Atrax®, Atranine A, designated as “A”); brompheniramine maleate (Dimegan®, Atranine B, designated as “B”); promethazine (Phenergan®, Atranine C, designated as “C”); and dexchlorpheniramine maleate (Polaramine®, Atranine D, designated as “D”).

These four compounds were added at different concentrations to cultures of various malignant cell lines and normal cells. The cell lines used for these tests were the following:

| | |
|---|---|
| K562 | myeloid leukemia; |
| KS | revertant of K562 that exhibits reduced tumorigenicity; |
| U937 | premonocytic leukemia; |
| US4 | revertant of U937 that exhibits reduced tumorigenicity; |
| Jurkat | T lymphocyte, acute leukemia of T cells; |
| T47-D | breast cancer, ductal carcinoma; |
| MCF7 | breast cancer, ductal carcinoma; |
| BT20 | breast cancer, carcinoma of the mammary glands; |
| LoVo | colorectal adenocarcinoma; |
| 184B5 | breast, immortalized, non-tumorigenic cells of luminal |
| | epithelium; |
| MCF10A | breast, immortalized, non-tumorigenic luminal epithelium |
| | cells; and T and B cells freshly isolated from three healthy |
| | donors. |

The tests on various cell lines were performed according to the following procedures.

5.1.1. Leukemic Cell Lines

All leukemic cell lines were grown and used in logarithmic phase. After one day of treatment, the cells were isolated, counted and diluted in a regular growth medium to provide the cell density of about 75×10³ cells/ml for reading on plates at 48 hours after the treatment, and 9.375 cells/ml for reading at 144 hours after the treatment.

Several dilutions of each compound tested were added to the wells (12 wells per plate, TPP), and 1 ml of cells from the parent solution was added per well after a line had been completed. The dilutions were made using the culture medium, and following dilutions were tested: control, which contained no compound of this invention; 1:100; 1:1,000; 1:2,000; 1:5,000; and 1:10,000. The starting concentrations for these dilutions were: 50 mg/ml for compound A; 10 mg/ml for compound B; 25 mg/ml for compound C; and 5 mg/ml for compound D. The results were analyzed by manual counting and Alamar reduced assay.

5.1.2. Lymphocytes from Healthy Donors

The blood from healthy donors was collected on citrate and diluted 1:1 with 0.15M NaCl. The dilution (6 ml) was loaded onto 3 ml of lymphoprep® (Nycomed), and the mixture was centrifuged at 800 g for 30 minutes at room temperature. The white cells were isolated and washed with RPMI1640 and 10% FBS. The white cells were diluted to provide 450,000 cells/m in RPMI1640/10% FSB medium. The tests for the compounds of this invention were performed according to the same procedures described in Section 5.1.1.

5.1.3. Adherent Cells of Breast and Colon

The cells were grown on their respective propagation medium and seeded 24 hours before the compounds are added. The cells were trypsinized, and 50,000 cells/well and 10,000 cells/well were seeded in order to read the plates at 48 hours and 144 hours after being treated by the compounds, respectively.

On the day of the treatment, the medium was replaced by 10 ml each of series dilution of the compounds, consisting of: control, which contained no compound of this invention; 1:100 dilution; 1:1,000 dilution; 1:2,000 dilution; and 1:5,000 dilution. The starting concentrations for these dilutions were: 50 mg/ml for compound A; 10 mg/ml for compound B; 25 mg/ml for compound C; and 5 mg/ml for compound D. The dilution was made using the growth medium.

5.1.4. Results

A tested compound is regarded as being active in cytotoxicity where the percentage of surviving cells after the treatment with the compound is less than 30%.

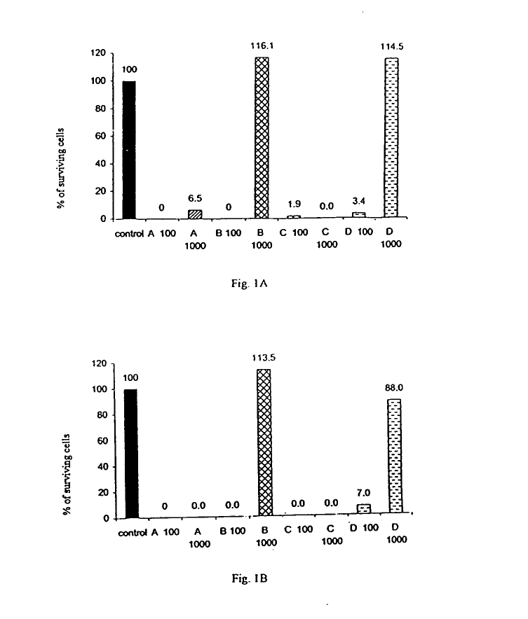

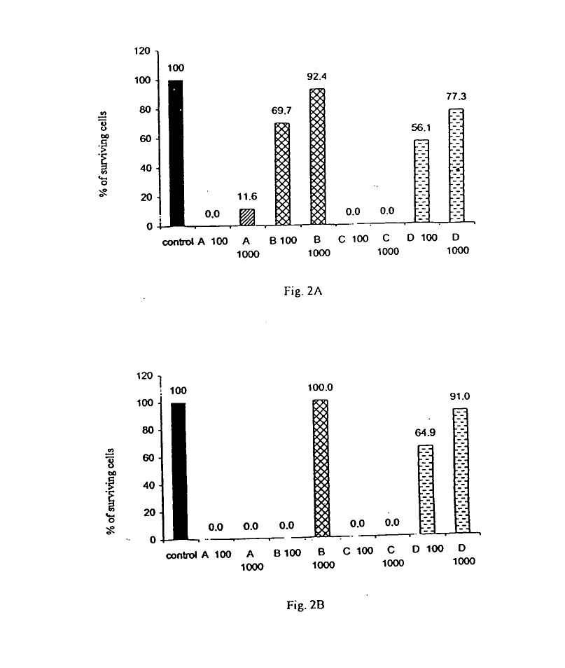

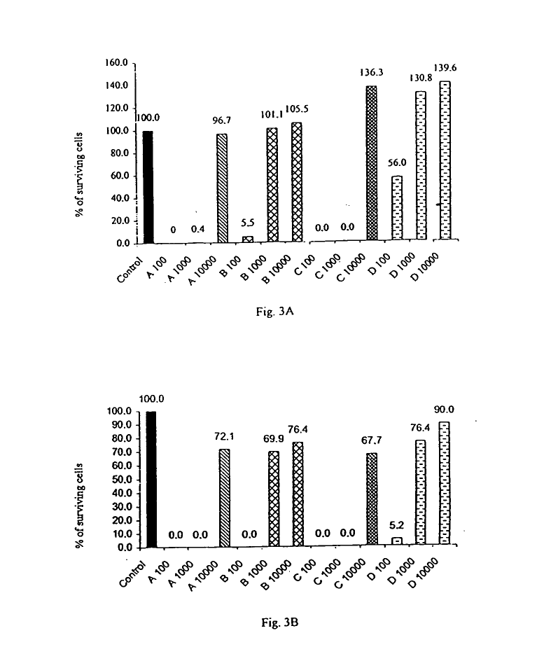

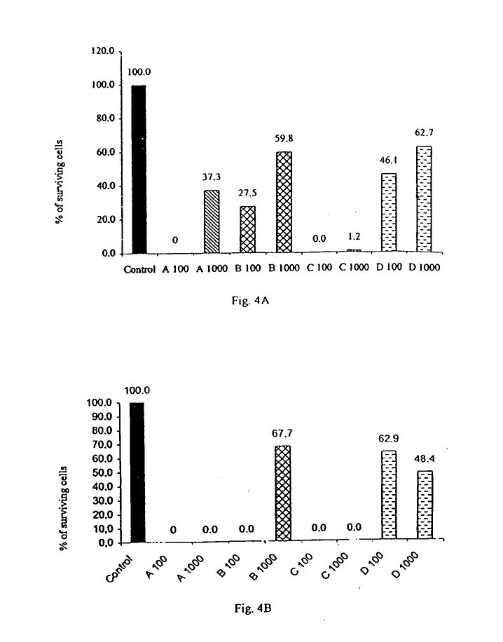

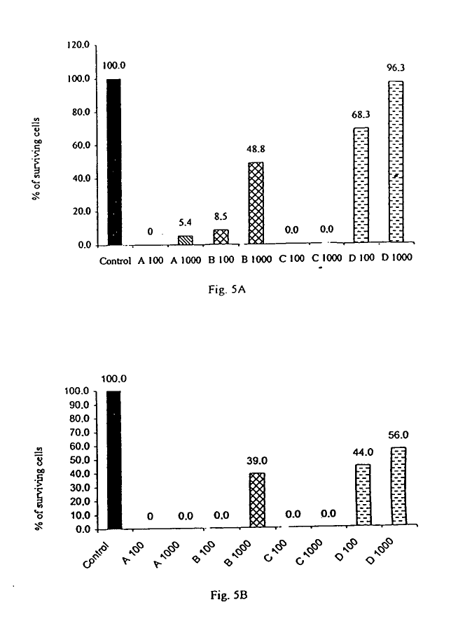

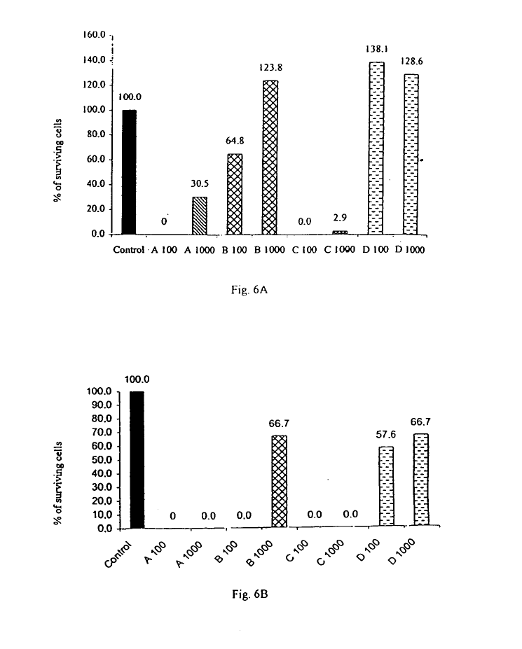

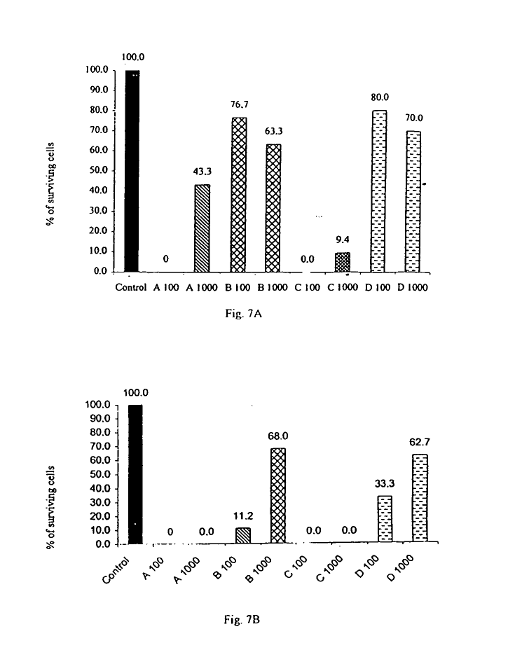

It was shown that compounds A and C exhibit a very good cytotoxicity, i.e., almost all the cancerous cells were destroyed, when used at dilutions of from 1:100 to 1:1,000. These compounds have been found effective when used in various cell lines as shown in FIGS. 1 through 7. Namely, these cell lines include K562 (FIG. 1), U937 (FIG. 2), Jurkat (FIG. 3), T47-D (FIG. 4), MCF7 (FIG. 5), BT20 (FIG. 6) and 184B5 (FIG. 7). In all of these figures, the “A” figures (e.g., FIG. 1A) show the results taken at 48 hours after the treatment, and the “B” figures (e.g., FIG. 1B) show the results taken at 144 hours after the treatment. Compounds A and C exhibited little cytotoxic activity when the tests were carried out using dilutions greater than 1:1,000, especially at about 1:10,000 dilution level. In all of the cell lines tested, compounds B and D were found to have little or no cytotoxic activity.

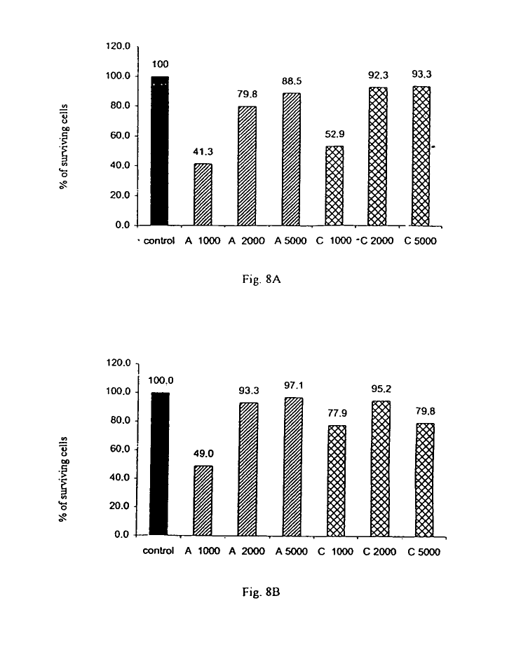

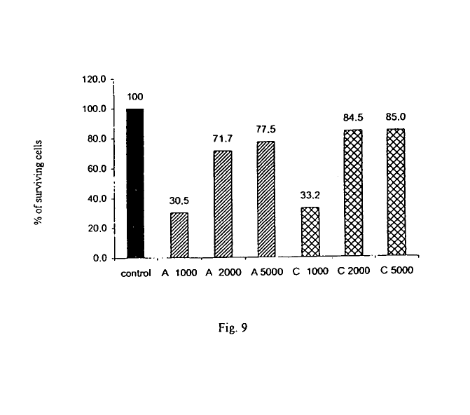

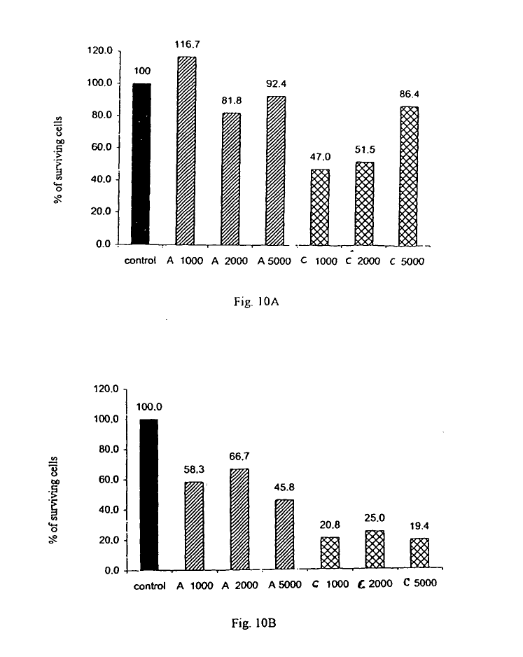

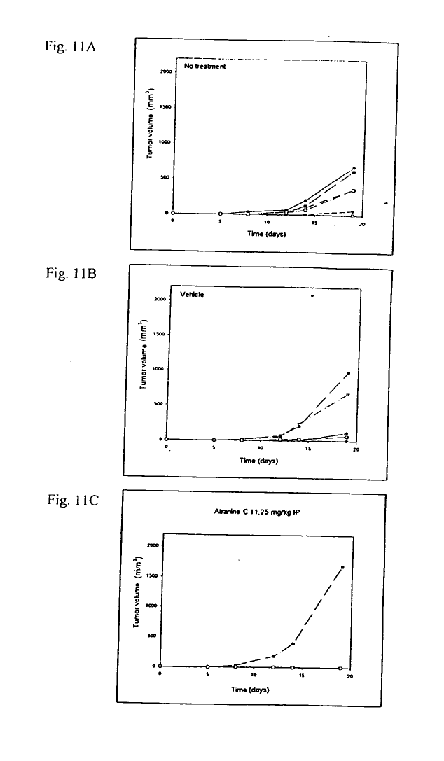

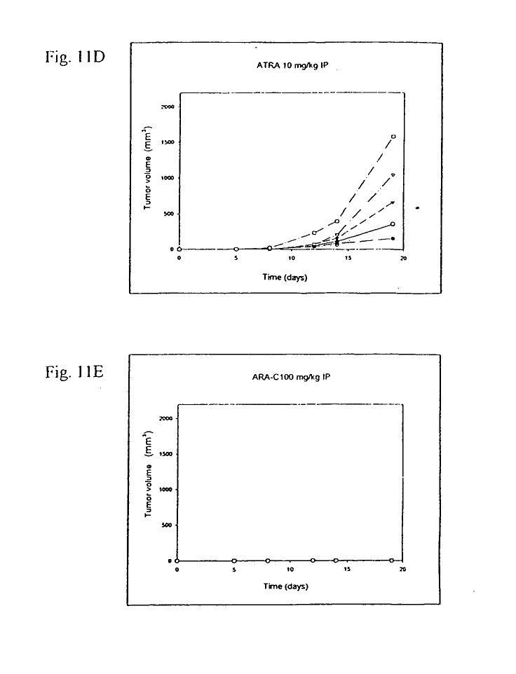

FIGS. 8A, 8B and 9 show the results obtained from these tests using the lymphocytes from three different healthy donors. As shown in these figures, the surviving cell counts from these lymphocytes at 48 hours after the treatment with compound A or C, even at 1:1,000 dilution level, are substantial. This result shows that compounds A and C exhibit a differential effect on the healthy and cancerous cells.

In order to demonstrate that this phenomenon is not arising from a general cytotoxicity of compounds A and C, assays were carried out on LoVo cancer cells, which are resistant to the cytopathic effect of the parvovirus HI. FIG. 10A shows that LoVo cancer cells are totally resistant to the antihistamines. This result indicates that the cytotoxicity of compounds A and C are not non-specific.

### 5.2. Example 2

### Antineoplastic Activity of Promethazine

Ten million U937 cells were subcutaneously injected into scid/scid mice. One injection was carried out per each of the 6 mice tested per set. The excipient or the compounds tested were administered intraperitoneally on day 1, i.e., the day of U937 cell line inoculation, of the experiment. The excipients or compounds were administered once daily. The tumor sizes were measured on day 5, day 8, day 12, day 14 and day 19 after the injection.

As a negative control, tumor development was monitored in six scid/scid mice, which received no treatment, and tumor was developed in five of the six mice tested (FIG. 11A). The pattern was similar for six mice treated only with excipients, where four out of six developed a tumor (FIG. 11B).

In contrast, as illustrated in FIG. 11C, only one out of six mice to which promethazine was administered daily at a concentration of 11.25 mg/kg developed a tumor. No tumor development was observed for the remaining five mice.

For a comparison purpose, All Trans Retinoic Acid (ATRA), which is known to have a certain level of antineoplastic activity, was administered to six scid/scid mice at a concentration of 100 mg/kg. As shown in FIG. 11D, all six mice developed a tumor. This result shows that promethazine has a much greater antineoplastic activity than ATRA.

As a positive control, ARA-C (cytarabine), which is a known antimitotic agent, was administered to six scid/scid mice at a concentration of 100 mg/kg. The administration of ARA-C resulted in tumor development in none of the six mice tested (FIG. 11E).

### 5.3. Example 3

### Other Effects of Hydroxyzine and Promethazine

5.3.1. TCTP Expression

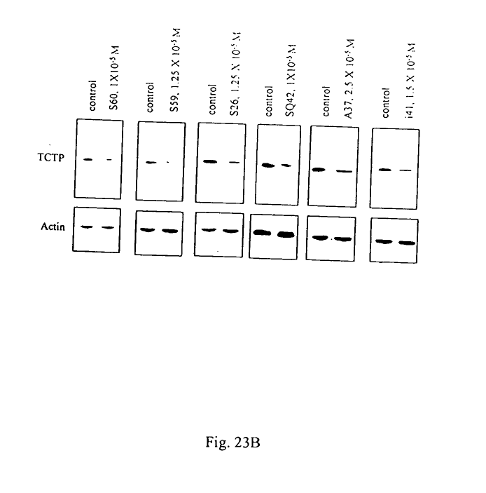

U937 cells were treated with various dilutions of hydroxyzine, brompheniramine and promethazine for 24 hours as denoted in FIG. 23A. Starting concentrations were: 50 mg/ml for hydrazine (“A”); 10 mg/ml for brompheniramine (“B”); and 25 mg/ml for promethazine (“C”). The proteins were isolated from these cells and loaded onto a gel for western blot analysis. A specific anti-TCTP (anti-HRF) antibody was used to visualize the location of TCTP. As shown in FIG. 23A, both hydroxyzine and promethazine inhibit the expression of TCTP, whereas brompheniramine exhibits little effect on the expression of TCTP. The level of actin in equal loading of extract was visualized by antiactin antibody (Santa Cruz Biotechnology). No substantial difference between the level of actin in treated and untreated cells was observed. This result shows that hydroxyzine and promethazine selectively act on the TCTP expression.

5.3.2. Poly ADP-Ribose Polymerase (PARP) Expression

U937 cells were treated with various concentrations of hydroxyzine and promethazine for 24 hours. The proteins were isolated from these cells and loaded onto a gel for a western blot analysis. A specific anti-PARP antibody was used to visualize the location of PARP. The results showed that hydroxyzine and promethazine, as well as staurosporine (positive control), cleave PARP, indicating the induction of apoptosis.

### 5.4. Example 4

### Cytotoxic Effects of Various Compounds of the Invention

In order to test the cytotoxic effects of various compounds, a test was developed in a multi-well plate. Two leukemic cell lines, namely K562 and U937, and three breast cancer cell lines, namely MDA-DB231, BT20 and MCF7 were used for this test.

The cells were seeded at low density and were left for 24 hours to recover and reach a new logarithmic phase of growth. Various concentrations of the compounds being tested, ranging from 2×10⁻⁹ M to 2×10⁻⁴ M, were added in triplicate. The mixture was incubated for 6 days, during which approximately 4 doublings of the cell population occurred, to allow the cells to reach subconfluence.

The viability of the cultured cells after the treatment by the compounds was determined by quantifying the level of ATP since the level of ATP is directly proportional to the number of viable cells present in the culture. The ATP level was quantified by using CellTiter-Glo™ (Promega) luminescent test for cell viability.

From the results of this test, the compounds were classified into three groups. Such groups are: group I, encompassing the compounds exhibiting powerful cytotoxic effects at log-5 M; group II, encompassing the compounds exhibiting cytotoxic effects at log-5 M, but with a less degree than that of compounds in group I; and group III, encompassing the compounds that are highly toxic at log-4 M, but less toxic at log-5 M.

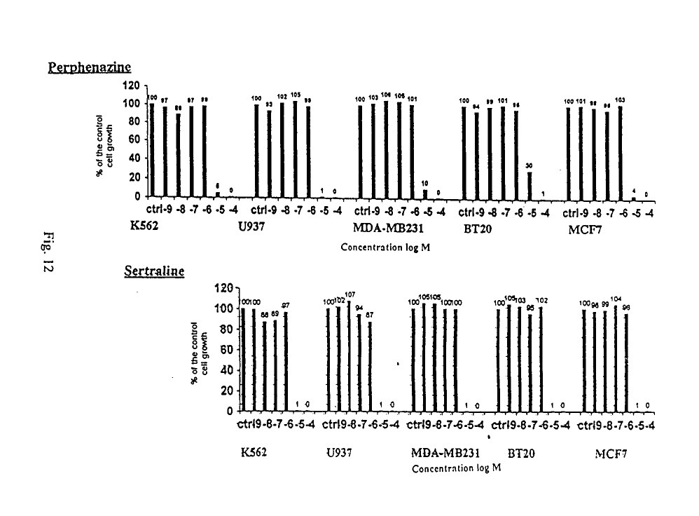

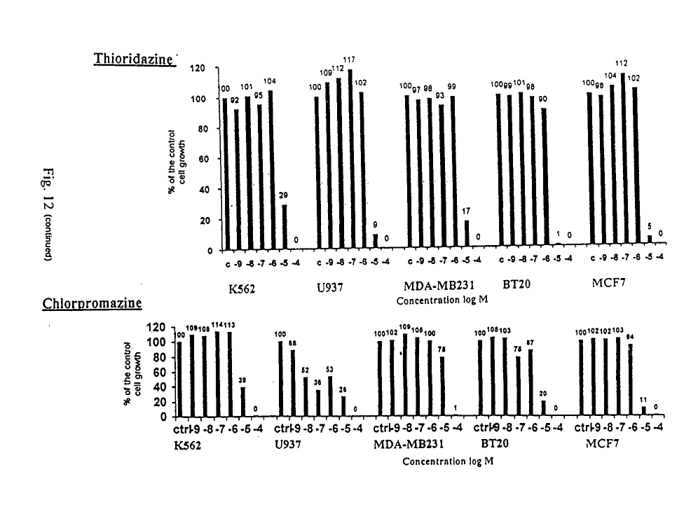

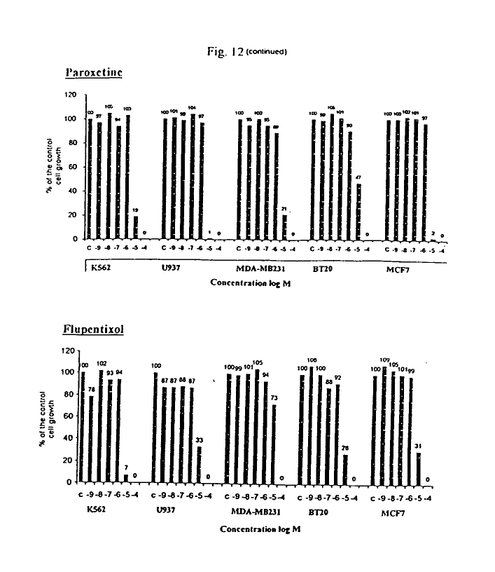

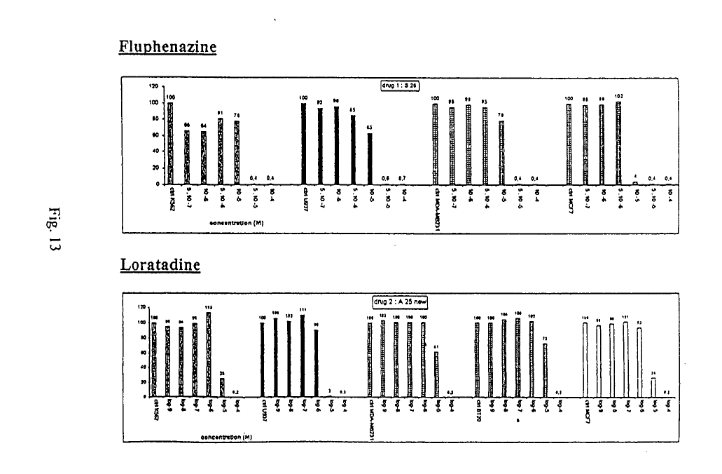

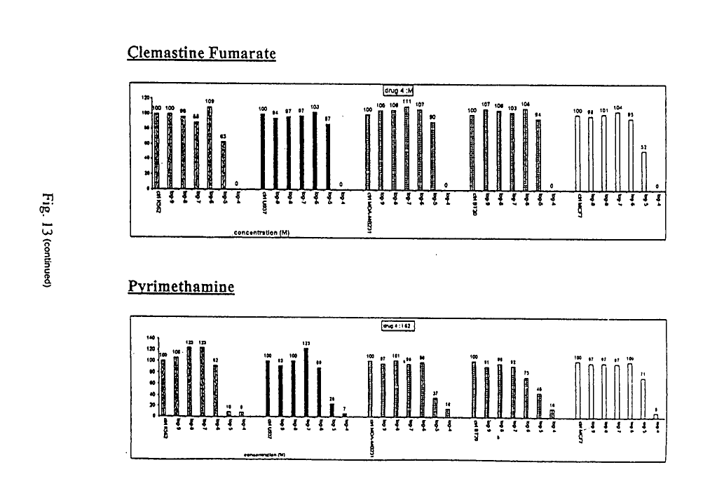

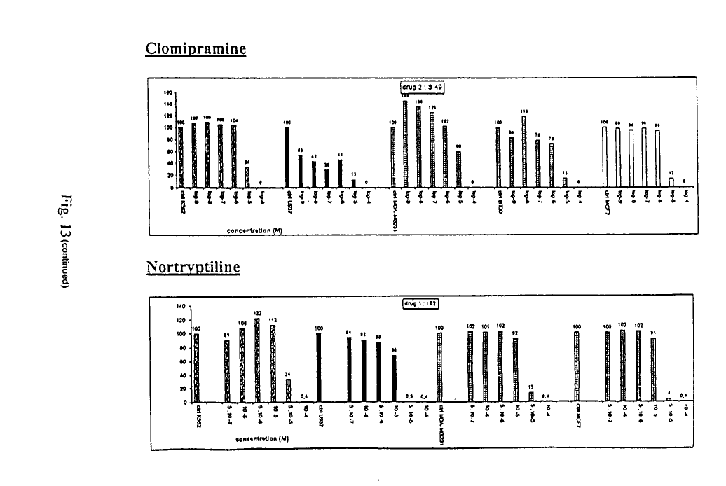

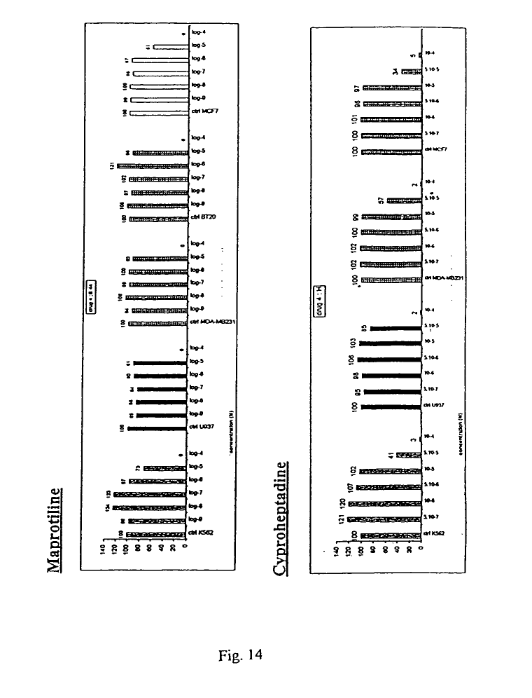

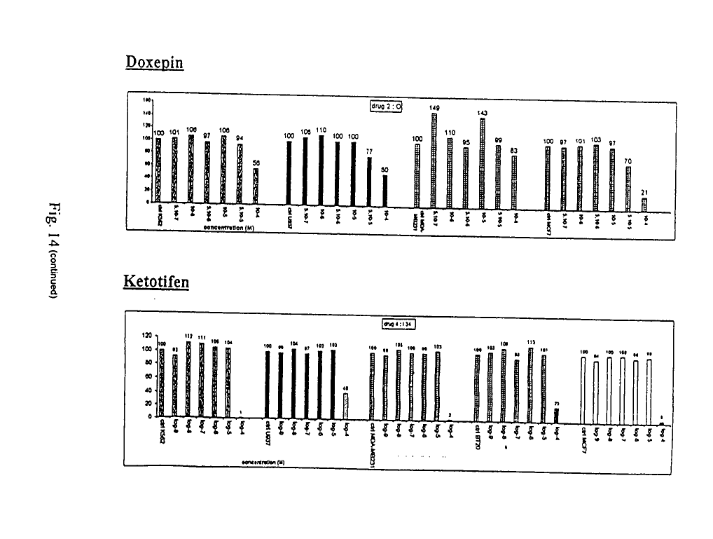

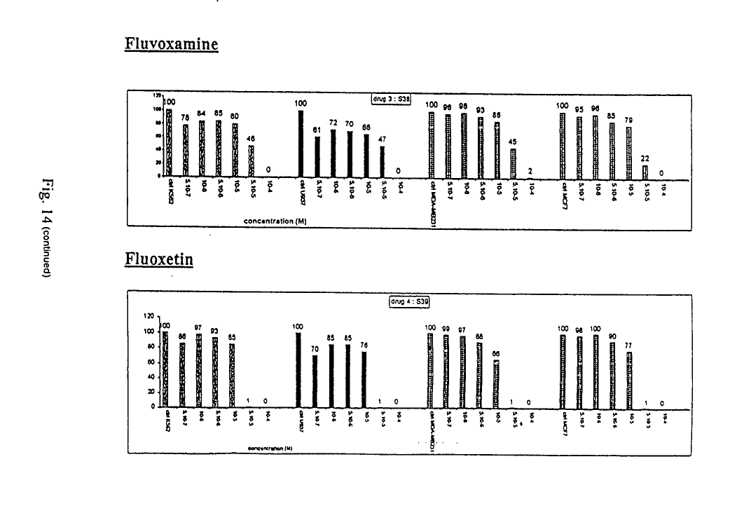

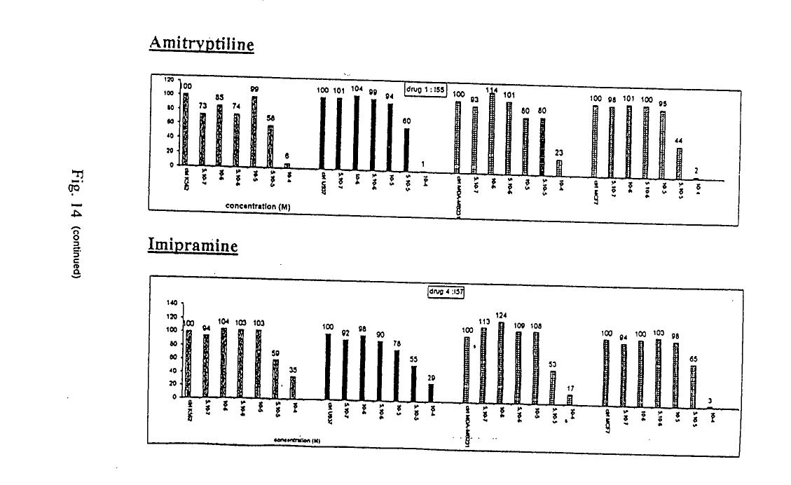

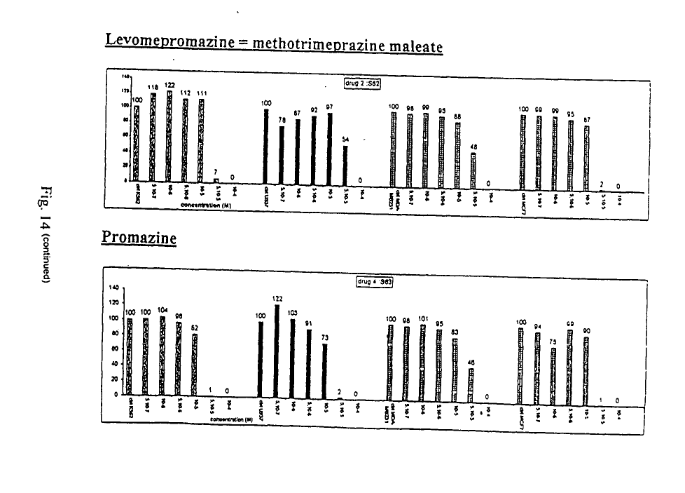

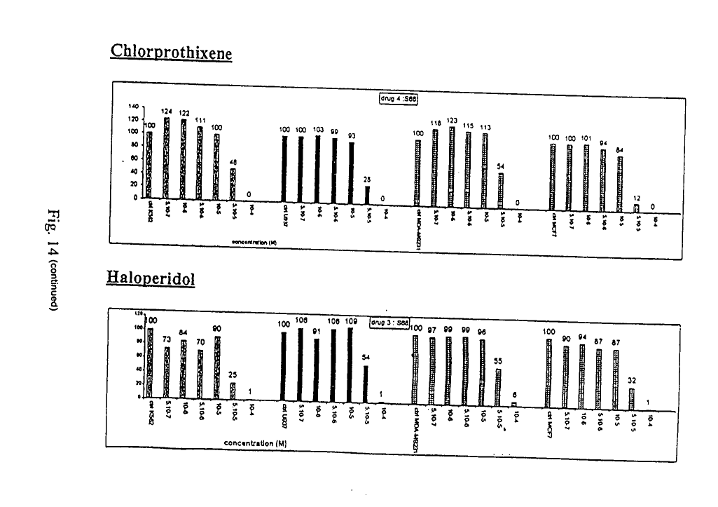

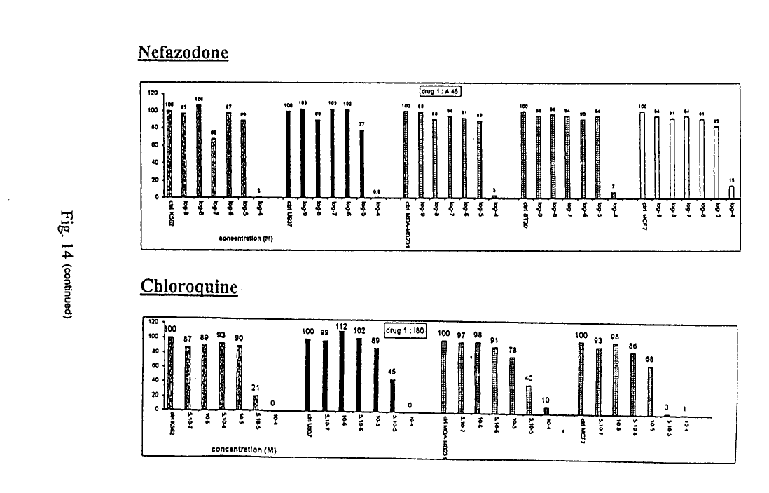

The compounds that can be classified as group I are perphenazine, sertraline, thioridazine, chlorpromazine, paroxetine and flupentixol. Their cytotoxic effects are shown in FIG. 12. The compounds of group II include fluphenazine, loratadine, clemastine, fumarate, pyrimethamine, clomipramine and nortryptiline. Their cytotoxic effects are shown in FIG. 13. The compounds of group III include maprotiline, cyproheptadine, doxepin, ketotifen, fluvoxamine, fluoxetine, amitryptiline, imipramine, levomepromazine, promazine, chlorprothixene, haloperidol, nefazodone and chloroquine. Their cytotoxic effects are shown in FIG. 14.

### 5.5. Example 5

### Cytopathic Activity of Various Compounds of the Invention

The cytopathic activity of the compounds of this invention, which are derived from histamine inhibitors, was determined as described in subsequent sections. Throughout these test, the following designation for the compounds being tested has been used: SQ42 for sertraline; S64 for perphenazine; A37 for Paroxetine; S59 for chlorpromazine; S60 for thioridazine; S26 for fluphenazine; and i41 for flupentixol.

5.5.1. Inhibition of Growth in U937 Cells

Various compounds derived from histamine inhibitors were tested in a liquid culture system on the U937 cell line. The results were expressed as % inhibition of growth as compared to negative control.

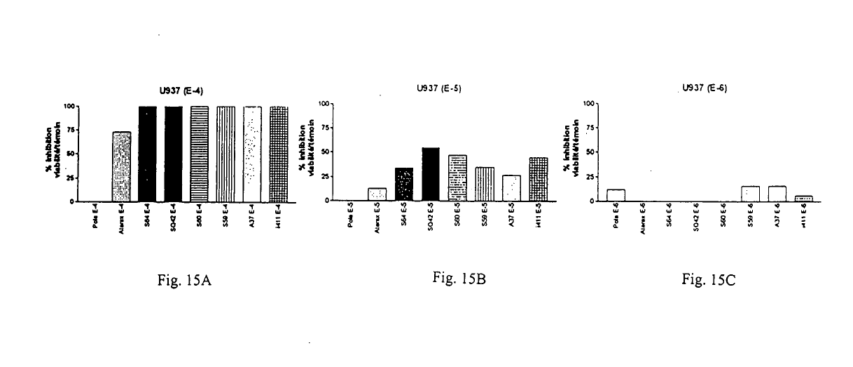

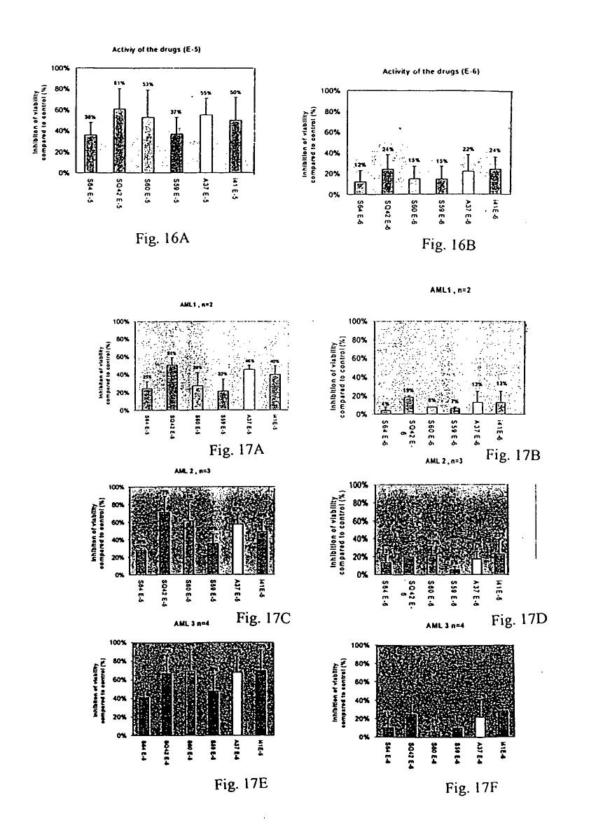

FIG. 15 shows the effects of hydroxyzine, S64, SQ42, S60, S59, A37 and i41 on the growth of U937 cells. FIGS. 15A through 15C correspond to the effects shown by these compounds at varying concentrations ranging from 10-4 to 10-6 M. The number is representation of cell death in excess of the usual death of 20-30% observed in the negative control.

5.5.2. Inhibition of Viability of Fresh Leukemic Cells

Six anti-histamine derivatives (S64, SQ42, S60, S59, A37 and i41) were tested for their effects on cell viability on blastic cells from AML patients. Mononuclear cells were isolated from peripheral blood of 15 patients having Acute Myeloid Leukemia (AML) using Ficoll-Hypaque centrifugation. Samples for AML 1 through 5, AML 1 being the least and AML 5 being the most differentiated, were obtained. Short term cultures were performed in RPMI 1640 with 15% FCS and 5% CO₂. Cells were incubated with or without 10- to 10 M anti-histamine derivatives. Polaramine was included as a negative control. Cell counts were assessed by trypan blue exclusion test at day 3 after the incubation. Morphological analysis was performed using Wright-Giemsa staining on cytocentrifuge preparations. The results were expressed as % inhibition of growth as compared to negative control.

As shown in FIGS. 16A and 16B, the % inhibition of cell viability varied from 34 to 61% when 10-5 M of these compounds was used and 11 to 24% when 10-6 M was used.

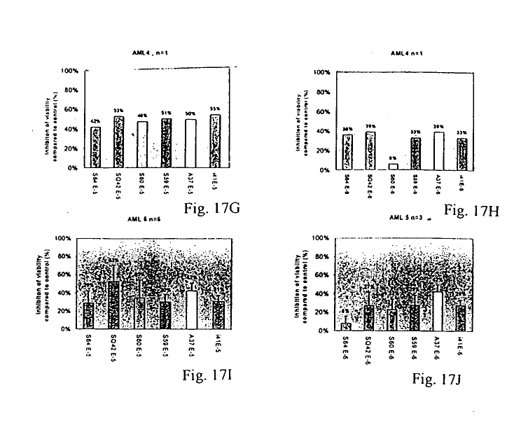

The inhibitory effects of these compounds were also measured ex vivo for each subgroup of AML, and the results are summarized in FIG. 17. As can be seen, these anti-histamine derivatives exhibit significant effects on the viability of fresh leukemic cells in vitro as well. The best result was obtained for AML 4 and 5, but the response was detectable in all the AML subtypes tested. The results obtained from various antihistamine derivatives are summarized in Table 1.

TABLE 1
In Vitro Percent Inhibition of Viability

| | | S64 | | SQ42 | | S60 | | S59 | | A37 | | i41 | | Hydroxyzine | | POL |
|---|---|---|---|---|---|---|---|---|---|---|---|---|---|---|---|---|
| ID | FAB | 10⁻⁵ | 10⁻⁶ | 10⁻⁵ | 10⁻⁶ | 10⁻⁵ | 10⁻⁶ | 10⁻⁵ | 10⁻⁶ | 10⁻⁵ | 10⁻⁶ | 10⁻⁵ | 10⁻⁶ | 10⁻⁴ | 10⁻⁵ | 10⁻⁵ |
| 1 | AML1 | 17 | 8 | 42 | 17 | 42 | 8 | 8 | 8 | 50 | 0 | 20 | 25 | 77 | 10 | |
| 2 | | 32 | 0 | 59 | 20 | 13 | 8 | 35 | 5 | 41 | 25 | 30 | 0 | | | |
| 3 | AML2 | 31 | 12 | 83 | 16 | 91 | 12 | 48 | 7 | 67 | 25 | 68 | 34 | | | |
| 4 | | 27 | 25 | 84 | 18 | 42 | 19 | 22 | 9 | 62 | 21 | 38 | 35 | 44 | 9 | |
| 5 | | 26 | 3 | 26 | 46 | 51 | 35 | 37 | 0 | 43 | 6 | 40 | 1 | | | |
| 6 | AML3 | 35 | 24 | 35 | 11 | 89 | 13 | 24 | 13 | 54 | 19 | 85 | 24 | | | |
| 7 | | 45 | 0 | 82 | 15 | 95 | 12 | 53 | 3 | 63 | 3 | 50 | 30 | | | |
| 8 | | 50 | 18 | 72 | 36 | 56 | 0 | 44 | 14 | 56 | 12 | 46 | 38 | | | |
| 9 | | 35 | 0 | 79 | 38 | 38 | 0 | 71 | 11 | 97 | 53 | 97 | 23 | | | |
| 10 | AML4 | 42 | 36 | 53 | 39 | 48 | 6 | 51 | 33 | 50 | 39 | 55 | 33 | | | |
| 11 | AML5 | 57 | 7 | 68 | 46 | 43 | 39 | 25 | 32 | 32 | 39 | 75 | 36 | | | |
| 12 | | 54 | | 73 | | 88 | | 51 | | 76 | | 55 | | | | |
| 13 | | 47 | 0 | 42 | 11 | 62 | 15 | 31 | 13 | 53 | 53 | 40 | 24 | | | |
| 14 | | 23 | 18 | 38 | 25 | 15 | 25 | 40 | 38 | 33 | 35 | 25 | 23 | 45 | 18 | 9 |
| 15 | | 17 | 8 | 78 | 0 | 27 | 0 | 20 | 17 | 41 | 9 | 27 | 16 | | | |

| | | | | | | | | | | | | | | |
|---|---|---|---|---|---|---|---|---|---|---|---|---|---|---|
| MEAN | 36 | 11 | 61 | 24 | 53 | 14 | 37 | 15 | 55 | 24 | 50 | 24 | 55 | 12 |

5.5.3. Inhibition of Viability of Various Cell Lines

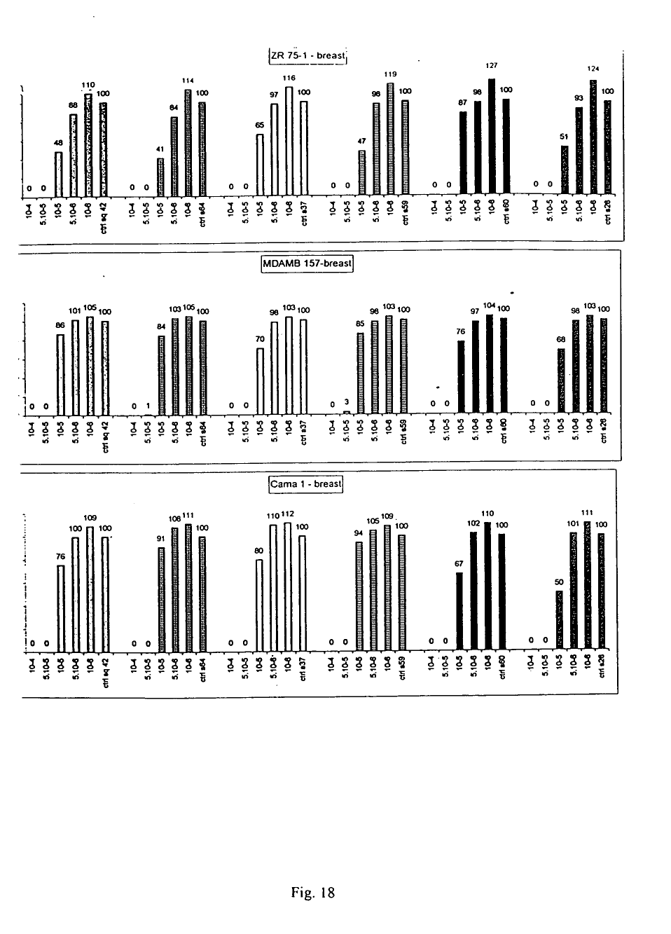

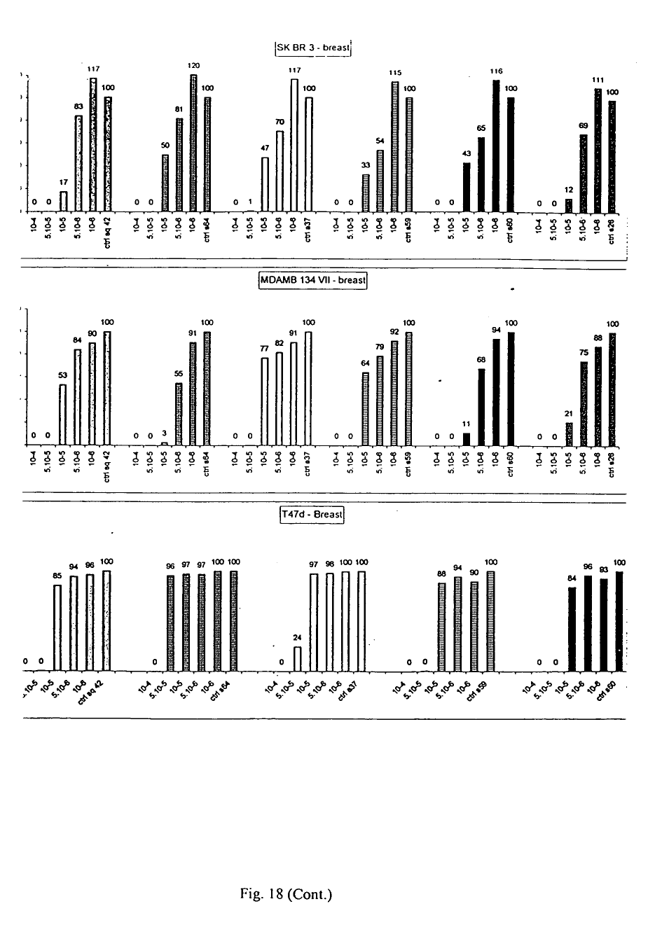

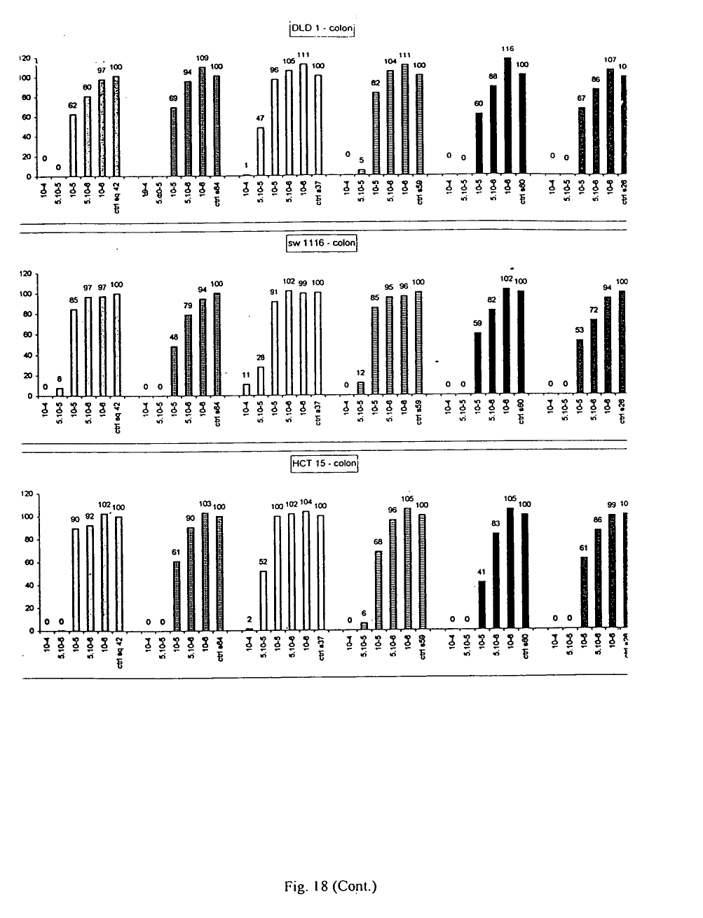

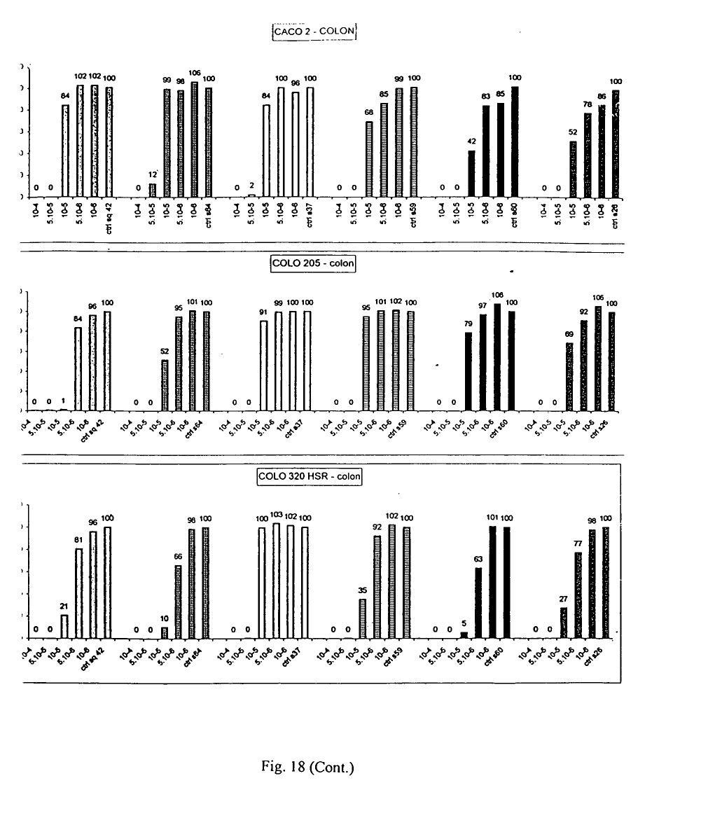

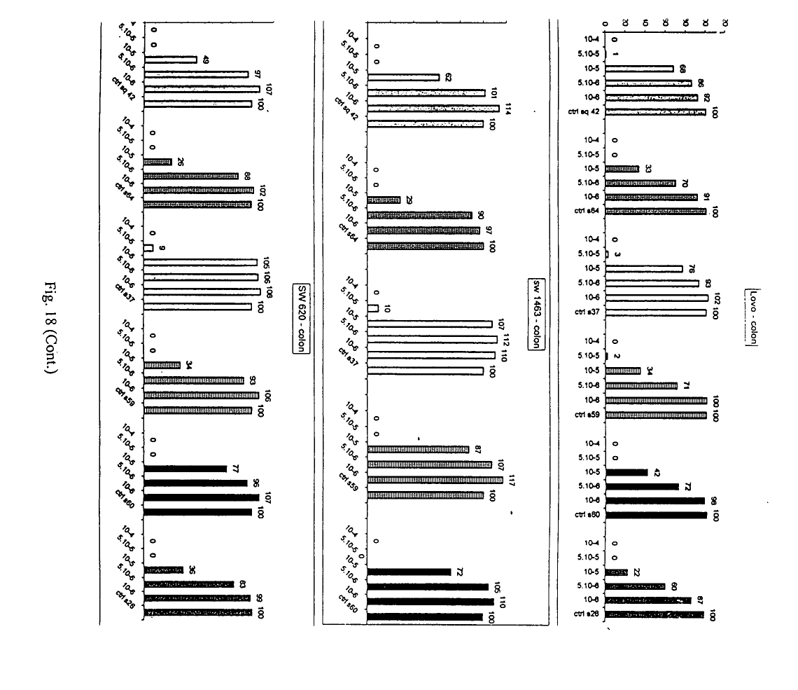

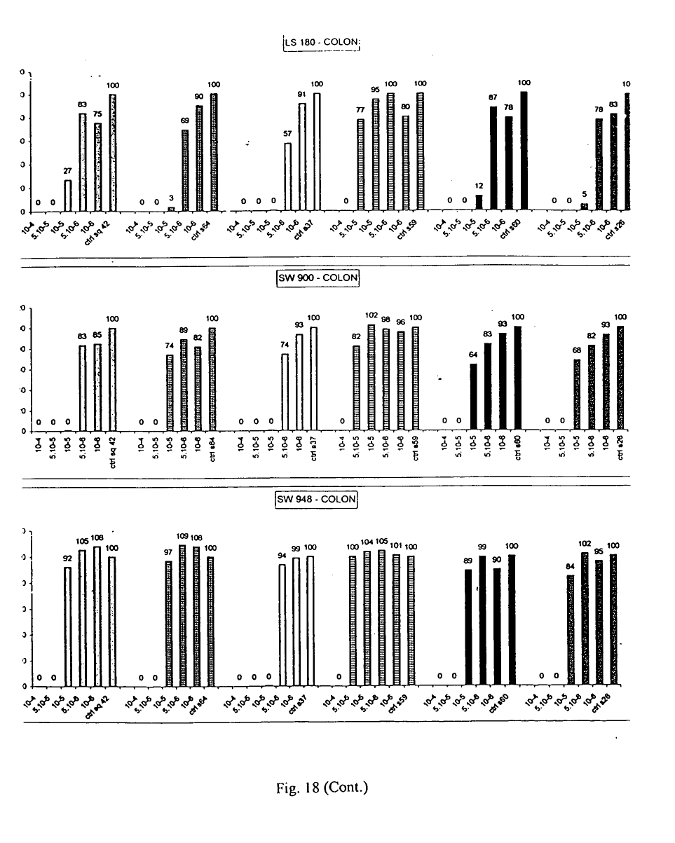

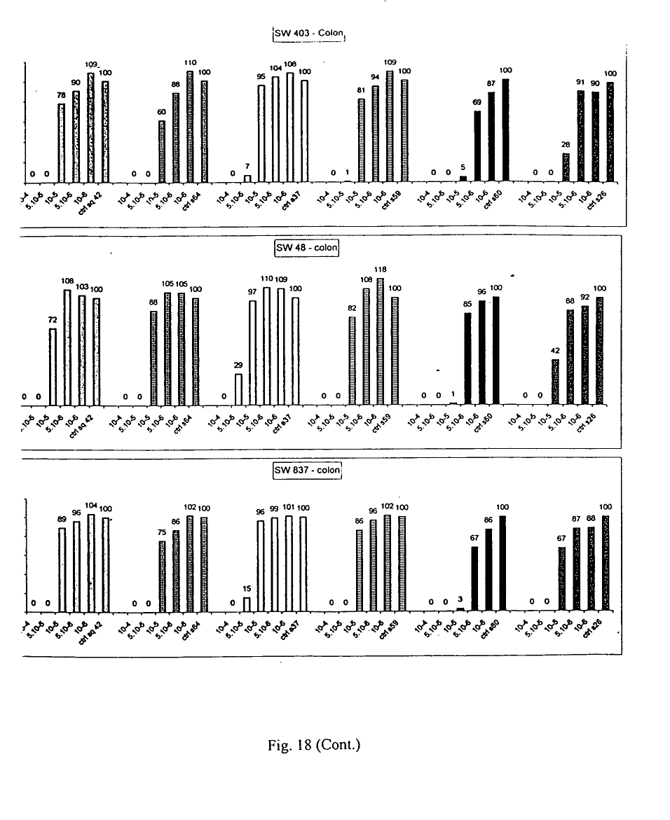

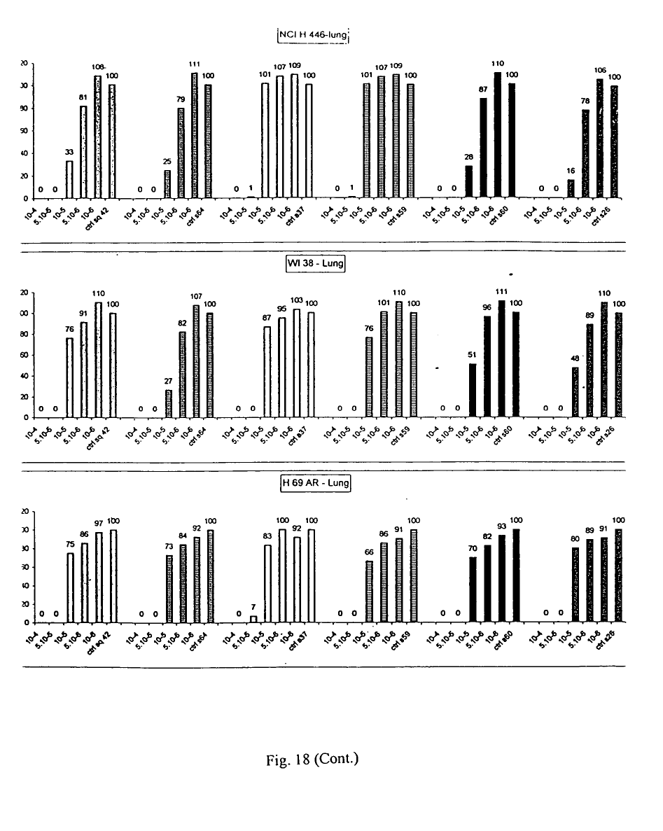

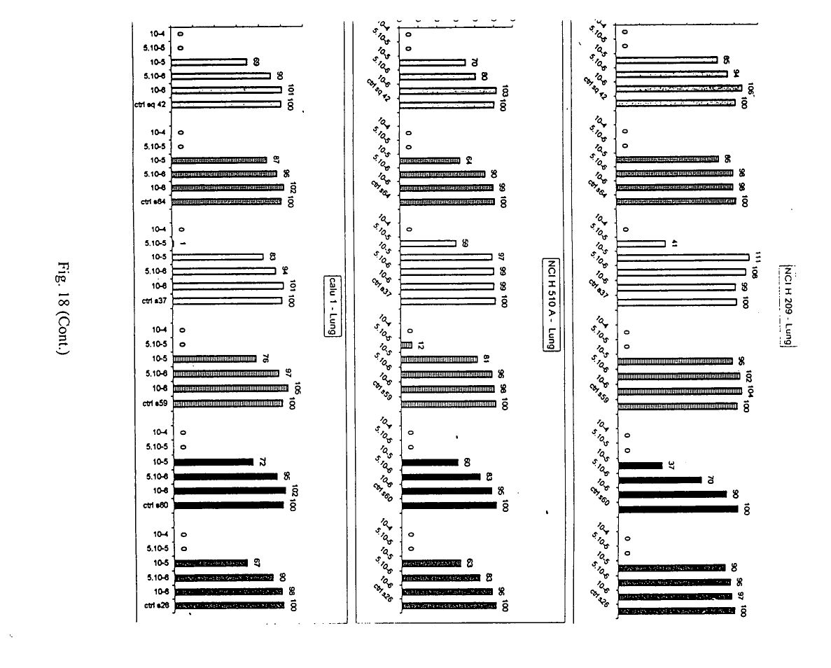

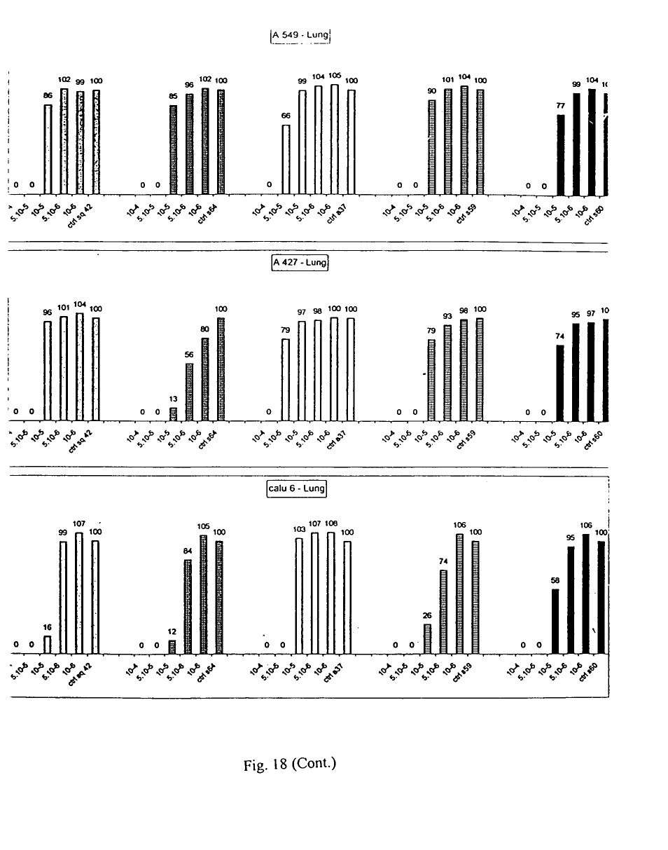

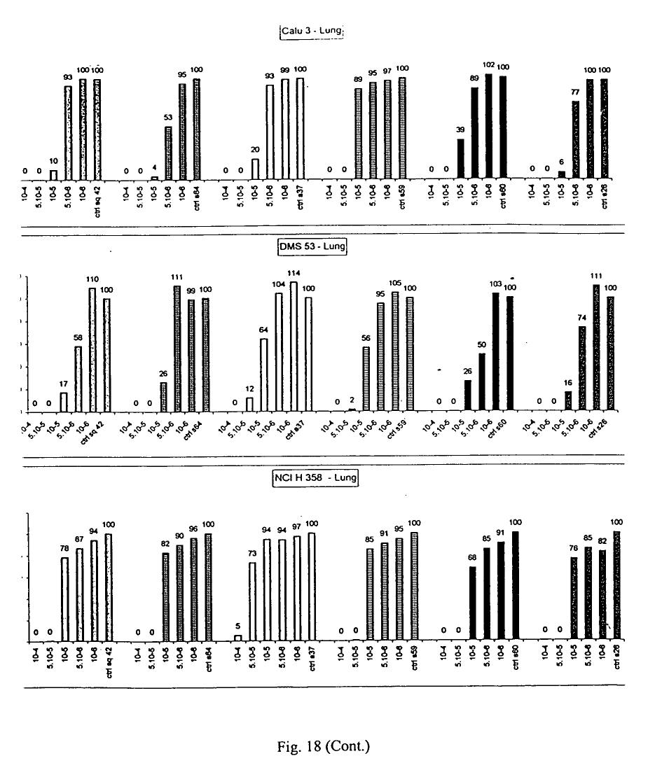

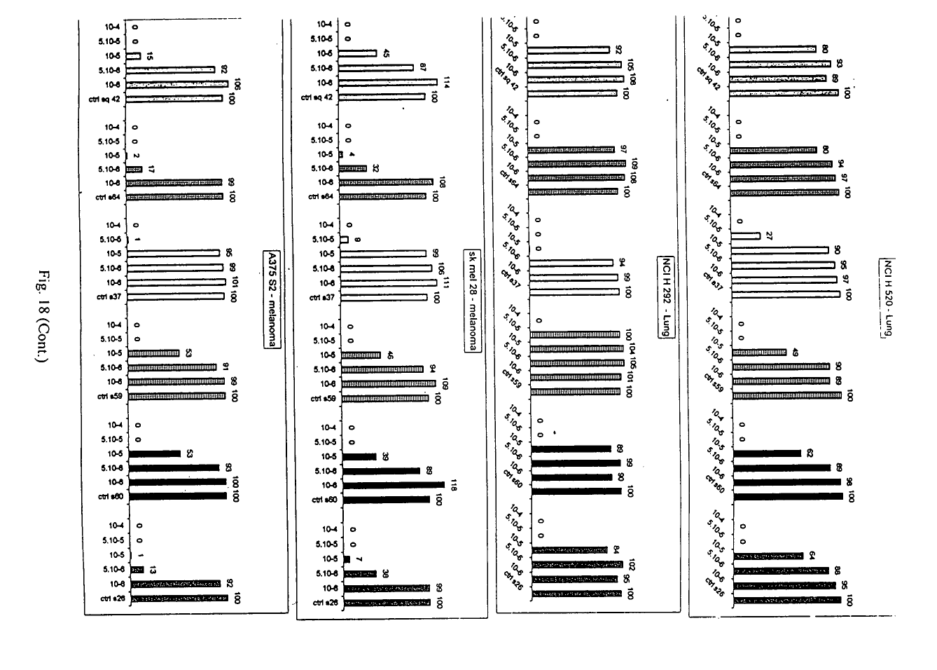

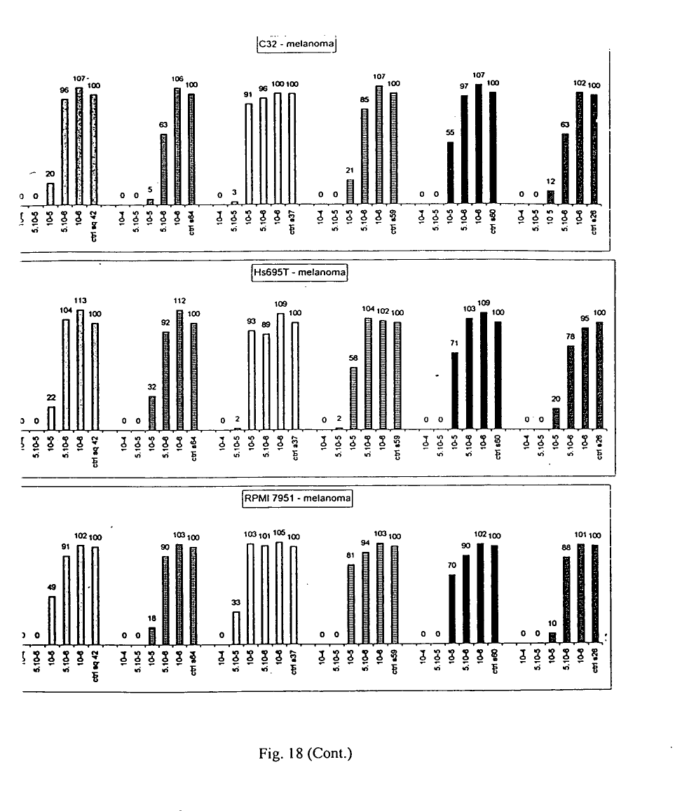

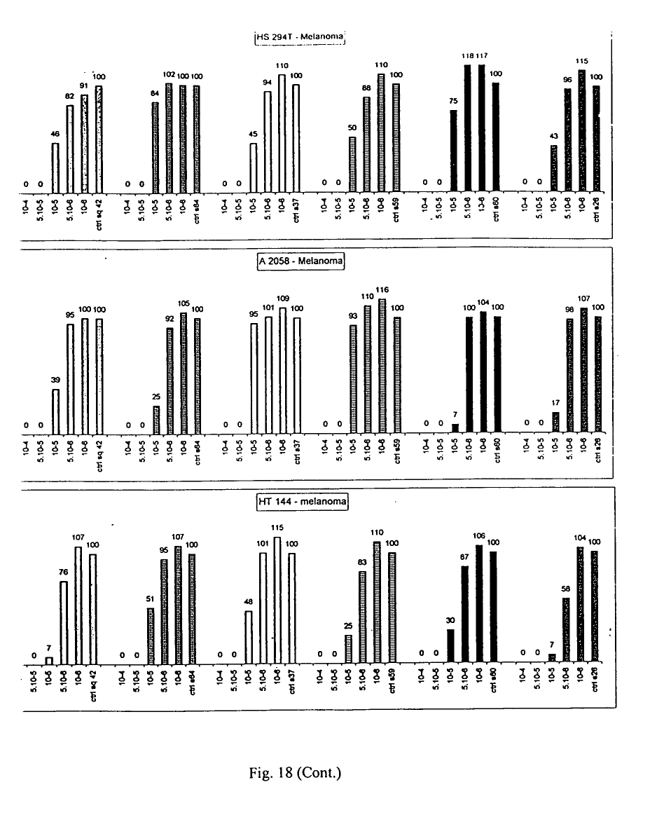

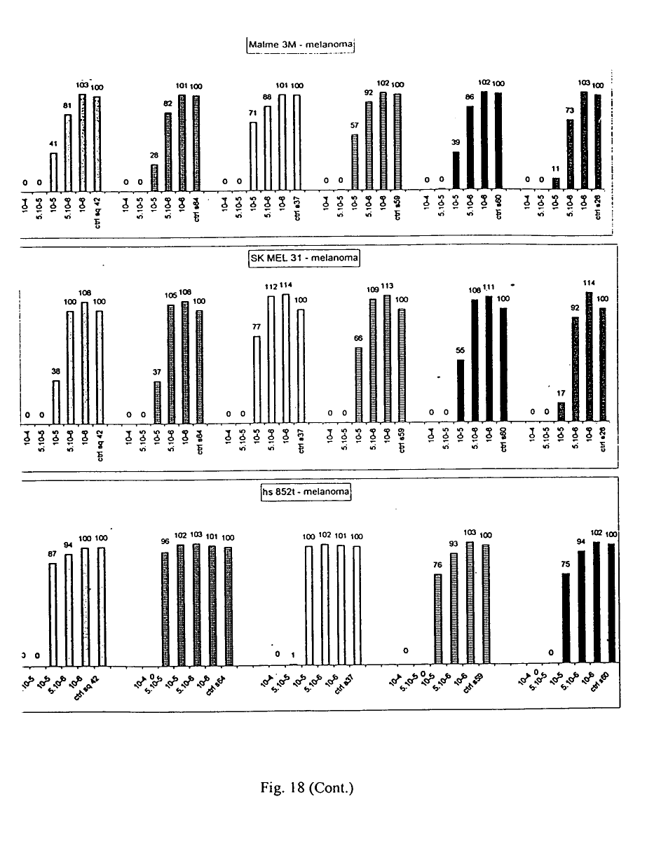

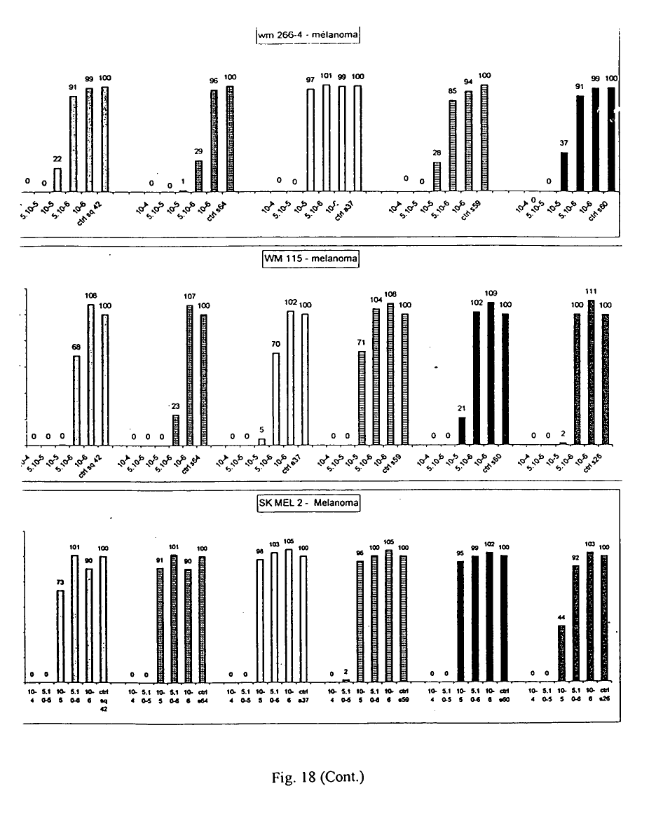

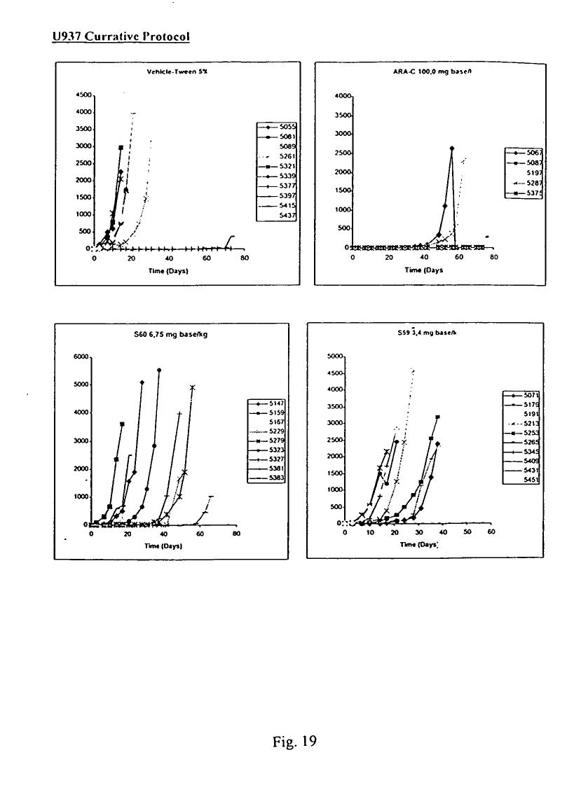

An assay in a multi-well plate was designed to assess the cytopathic effect of various antihistaminic or structurally/functionally related compounds. The compounds tested were SQ42, S64, A37, S59, S60 and S26. The compounds were prepared in their respective vehicle solution at a concentration of 1×10⁻² M. Following sterilization by filtration, the compounds were diluted to yield the concentrations as denoted in FIG. 18.

Various cell lines derived from breast cancer, colon cancer, lung cancer and melanoma were used in this assay. The cell lines used were:

Breast cancer cell lines consisting of ZR 75-1, MDA-MB 157, Cama-1, SKBR3, MDA-MB 134VII and T47 D;

Colon cancer cell lines consisting of DLD-1, SW1116, HCT-15, CACO-2, COLO-205, COLO-320-HSR, LOVO, SW-1463, SW-620, LS-180, SW-900, SW-948, SW-403, SW-48 and SW-837;

Lung cancer cell lines consisting of NCI-H-446, WI38, H-69-AR, NCI-H-209, NCI-H-510A, CALU-1, A549, A427, CALU-6, CALU-3, DMS-53, NCI-H-358, NCI-H-520 and NCI-H-292; and

Melanoma cell lines consisting of SK-MEL-28, A375-S2, C32, Hs695T, RPMI-7951, Hs-294T, A2058, HT-144, Malme-3M, SK-MEL-31, Hs-852T, WM-115 AND SK-MEL-2.

Cells were seeded at low density and unperturbed for 24 hours to allow recovery until they reach log phase. Various concentrations of the antihistaminic compounds were added in triplicate as indicated in FIG. 18. The contact with the antihistaminic compounds was maintained for 3 days, allowing the cells to reach sub-confluence. The viability of the cells after the treatment was determined by quantifying the level of ATP.

The level of ATP was quantified by using

CellTiter-Glo™ luminescent cell viability assay from Promega.

As illustrated in FIG. 18, all of the compounds tested showed efficacy in reducing the cell viability in a wide variety of cancer cell lines.

### 5.6. Example 6

### Effects on In Vivo Tumor Development

In this series of experiments, effects of various compounds of the present invention on tumor development were tested using two protocols. The first protocol is directed to the testing of curative effects of these compounds in different cell lines. In this protocol, cancerous cells are inoculated to the animals, and the inoculated animals were left without any further treatment until the development of tumors. As soon as the tumors became palable (4 mm³), the compounds of this invention were administered daily to the animals. Two different cell lines, i.e., U937 and MDA-MB-231 (breast cancer line), were used for this protocol to inoculate scid/scid mice.

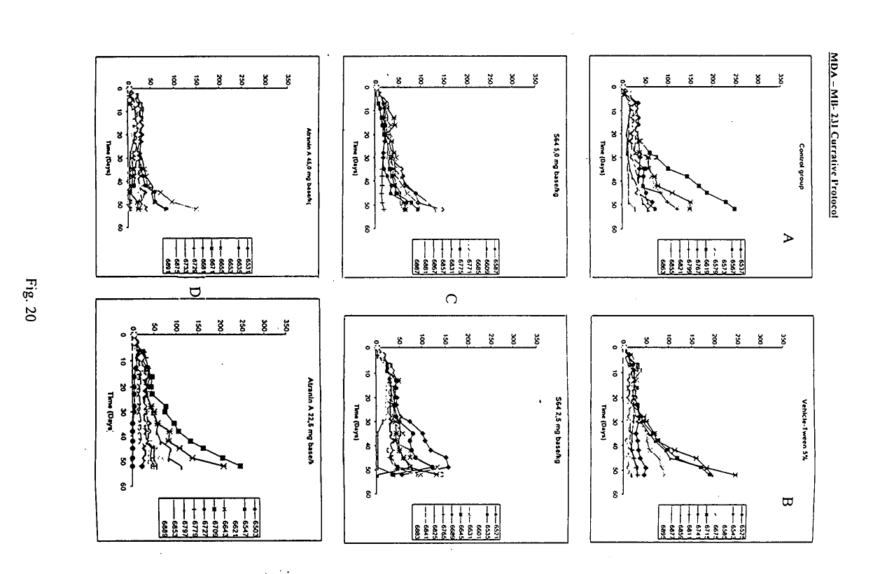

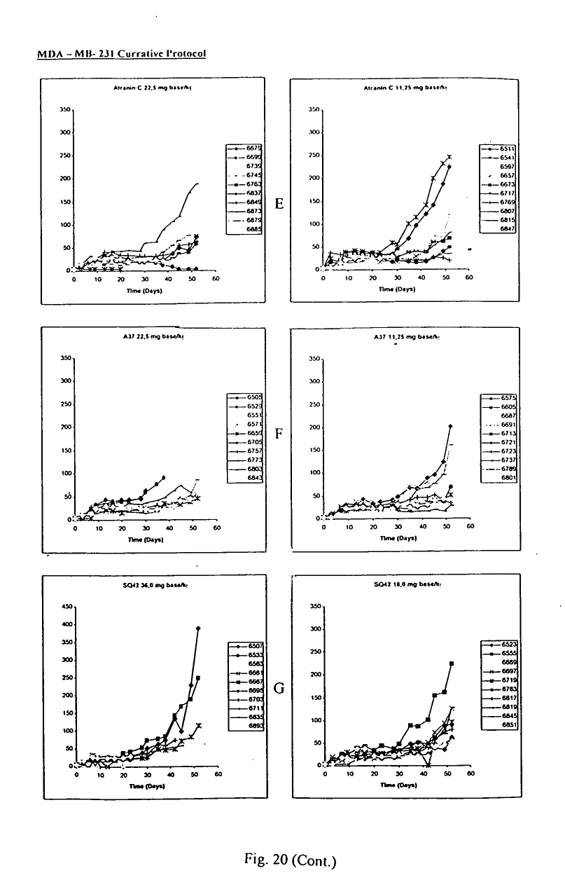

FIG. 19 illustrates the curative effects of compounds S60 and S59 in U937 cells. FIG. 19A is the result obtained from a negative control, wherein the animals were treated with excipient only. Eight out of 10 mice tested rapidly developed tumors. In a positive control, wherein the animals were treated with ARA-C, no tumor development was observed (FIG. 19B). As shown in FIGS. 19C and 19D, compounds S60 and S59 cause a delay in the tumor growth in U937 cells.

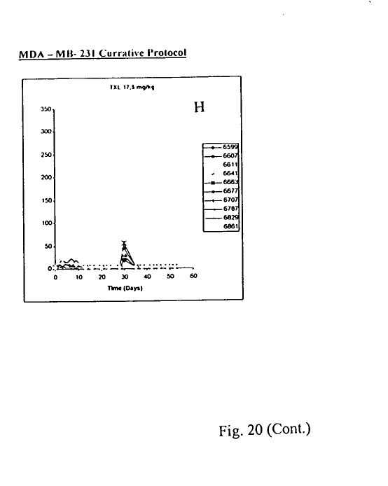

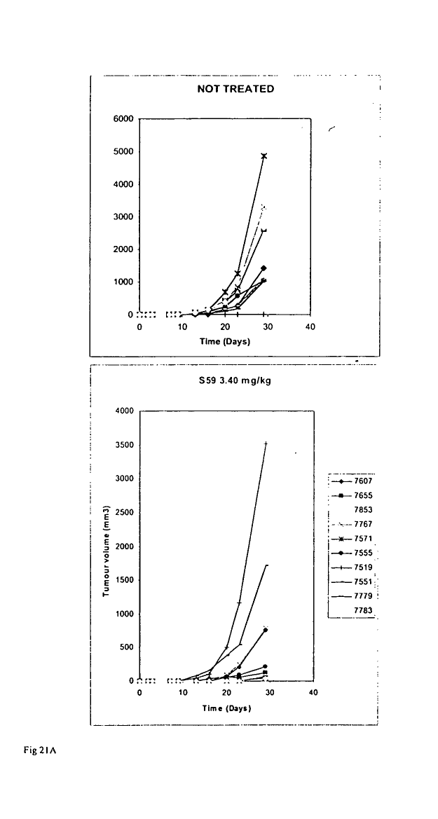

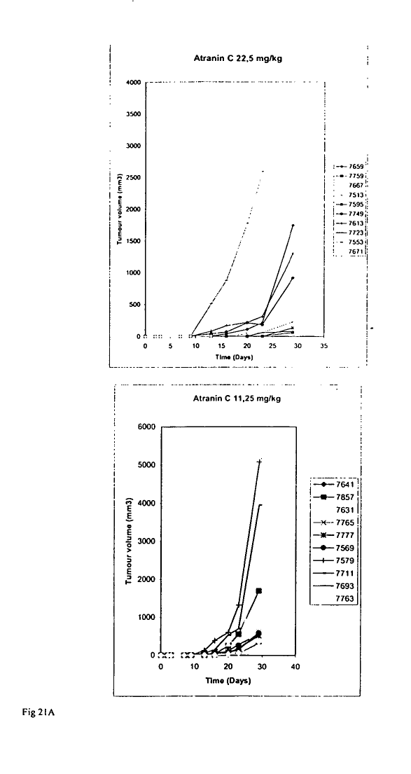

FIG. 20 illustrates the curative effects of the compounds of this invention in MDA-MB-231 cell line. In the animals that received no treatment, the major tumor development commences around day 30. Nine out of 10 mice developed tumors (FIG. 20A). FIG. 20B illustrates the result obtained from second control, wherein the animals were treated with excipient only. Eight out of 10 mice developed tumors.

As shown in FIGS. 20C through 20G, the compounds of this invention cause the decrease in tumor growth, at least at the higher concentration that was tested. In particular, it is noted that S64, at a concentration of 2.5 mg/kg, appears to cause a regression of the tumor volume (FIG. 20C). The same is also true for certain animals treated with promethazine, either at a concentration of 11.25 mg/kg or 22.5 mg/kg (FIG. 20E). For all other compounds tested, a general decrease in tumor growth was observed. In the case of compounds A37 and SQ42, higher dosage caused the death of certain test animals, but the general trend of decreased tumor growth remained the same with the surviving animals.

The second protocol was designed to test the preventive effects of the compounds of this invention on tumorigenesis. In this protocol, the animals have been treated with various compounds of the present invention on day 1. After more than 24 hours, tumoral cells were inoculated. The administration of the compounds of this invention was maintained daily. The cell line used for this test was U937.

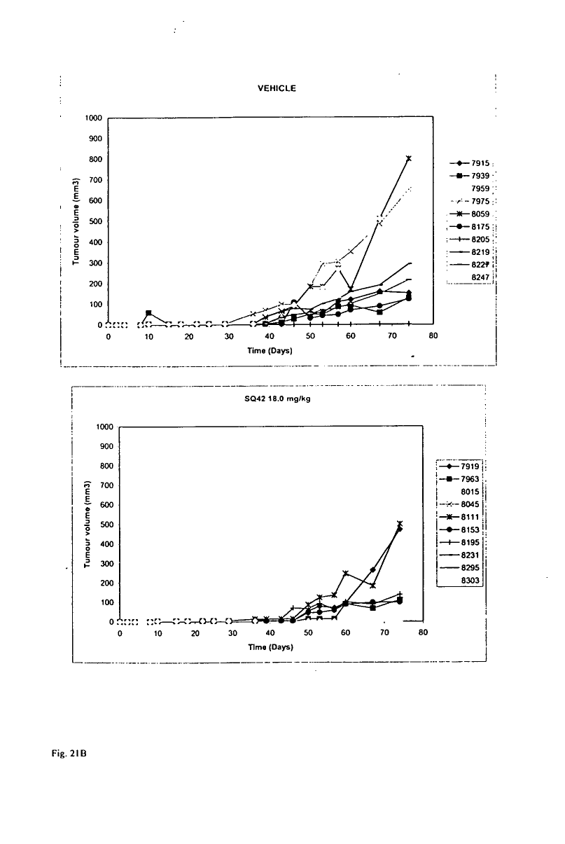

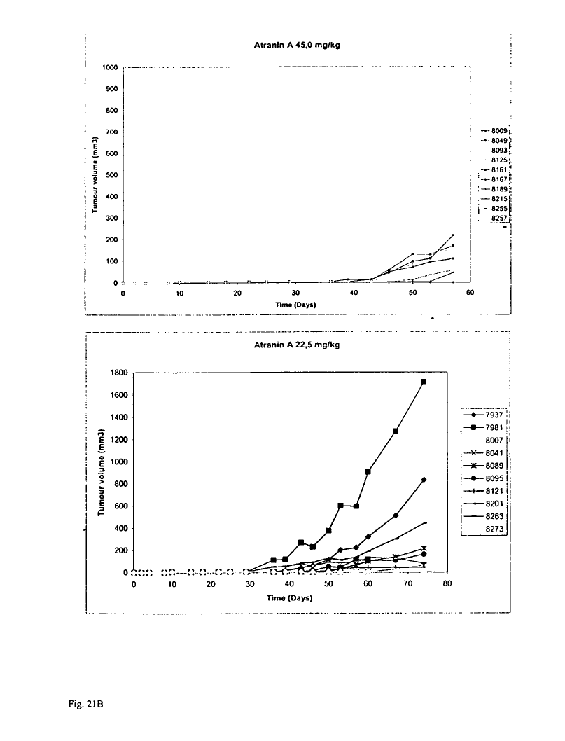

As illustrated in FIG. 21, compounds S49, S60 and promethazine caused a decrease in tumor growth in U937 cell line compared to the negative control. The treatment by A37 and SQ42 at higher dosage was toxic for the animals and caused the death of certain animals tested. However, it does appear that these compounds also cause the decrease in tumor growth.

### 5.7. Example 7

### TCTP Expression in Normal and Tumor Tissues

TCTP expression in tissues from various normal and cancerous organs was analyzed using a western blot analysis. The normal and cancerous tissues tested were obtained from liver, lung, pancreas, stomach, thyroid, laryngopharynx, skin, uterus, breast, cervix, ovary, testis, prostate and rectum. The proteins were isolated from these tissues and loaded onto a gel for western blot analysis. A specific anti-TCTP (anti-HRF) antibody was used to visualize the location of TCTP.

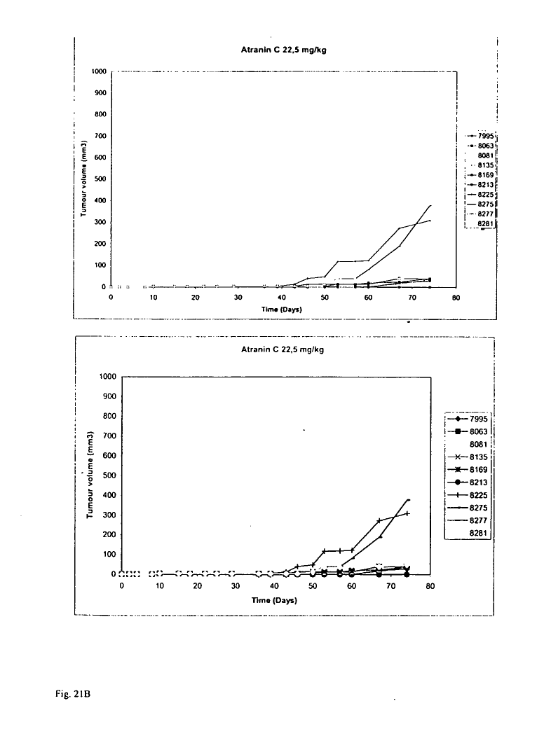

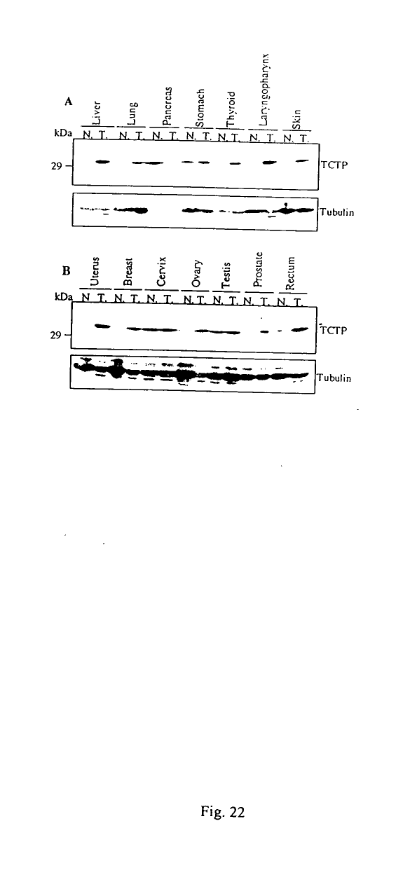

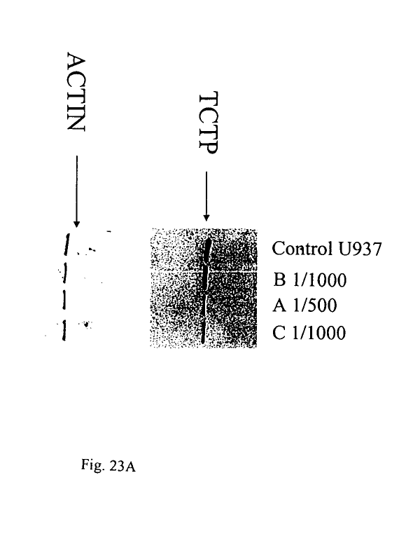

As shown in FIG. 22, most of the tissues tested exhibited an overexpression of TCTP in their cancerous state. This result confirms that TCTP is upregulated in a wide variety of tumor tissues.

### 5.8. Example 8

### TCTP Expression After Treatment by Various Antihistamines or Structurally/Functionally Related Compounds

The expression of TCTP following the treatment by S60, S59, S26, SQ42, A37 and i41 was examined by western blot analysis. The procedures were substantially the same as illustrated in Example 7, with the exception that the cells were treated with various antihistamines for 28 hours at 37° C. before the proteins were extracted.

FIG. 23 shows that down regulation of TCTP is exhibited in all cells treated with these antihistamines. The level of actin in equal loading of extract was visualized by anti-actin antibody (Santa Cruz Biotechnology). As shown in FIG. 23, no substantial difference between the level of actin in treated and untreated cells was observed. This result shows that these antihistamines selectively act on the TCTP expression.

### 5.9. Example 9

### Induction of Caspase 3 and 7 Activities

Activities of caspases are proportionally related to the apoptoses. Accordingly, another set of experiment was carried out to determine the activities of caspase 3 and 7 in U937 cells, following treatment with SQ42, S64, A37, S59, S60 and S26.

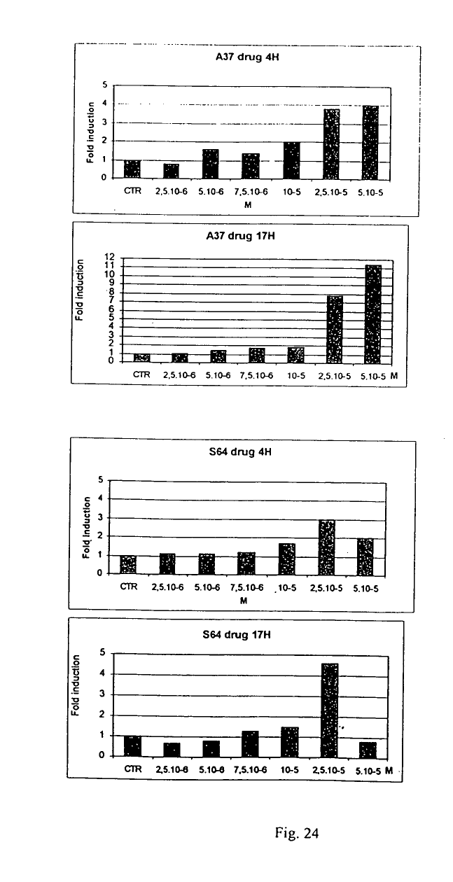

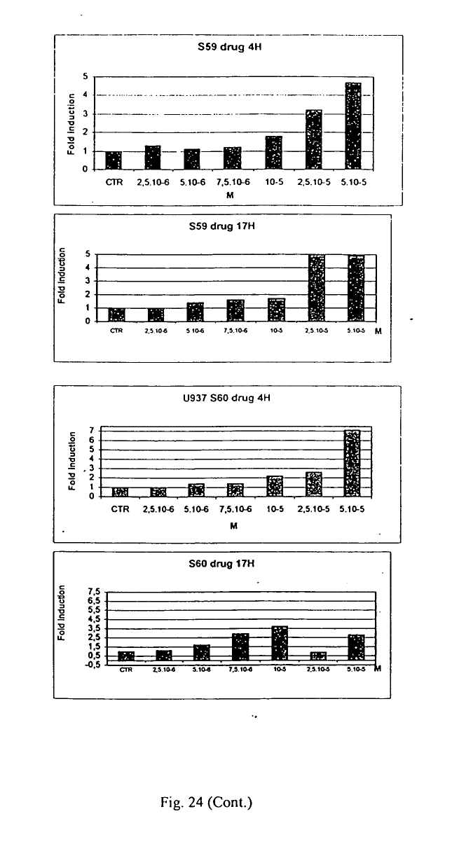

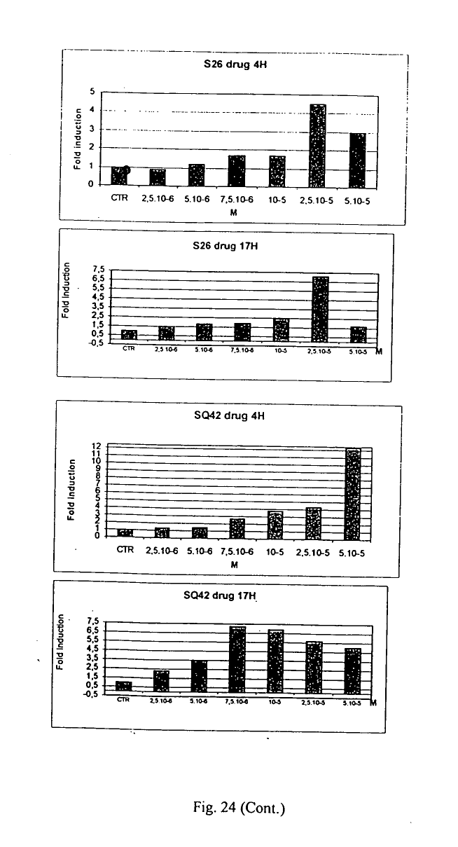

U937 cells were seeded at low density (50,000 in 100 μl medium) in white 96 well plates. The compounds of the invention or vehicle (control) were diluted in regular growth medium to a 2×C [can you clarify] solution and further 1:1 diluted with the cells to provide the final concentrations denoted in FIG. 24. After 4 or 17 hours, the plates were centrifuiged, and 100 μl was removed. 100 μl of Apo-One™ homogeneous caspase-3/7 assay mix was added. Solution was mixed for 30 seconds and further incubated for 1 hour at room temperature in the dark. The conversion of prefluorescent caspase substrate rhodamine 110-Z-DEVD to fluorescent product was determined by monitoring the fluorescence of the mixture. The fluorescence was measured on a fluorimeter at 499 nm excitation and 521 nm emission.

As shown in FIG. 24, treatment of U937 cells with the compounds of the invention resulted in an increase of cell death at either 4 or 17 hours. This result illustrates that these compounds cause the death of tumor cells in U937 cell line.

What is claimed is:

1. A method of inhibiting the growth of a cancer cell, which comprises contacting the cell with an antihistaminic agent or a structurally/functionally related compound, or a pharmaceutically acceptable salt, prodrug, solvate, hydrate or clathrate thereof.

2. The method of claim 1, wherein the antihistaminic agent or structurally/functionally related compound is perphenazine, sertralin, thioridazine, chlorpromazine, paroxetine, flupentixol, fluphenazine, hydroxyzine or promethazine.

3. A method of treating cancer, which comprises administering to a patient in need of such treatment a therapeutically effective amount of an antihistaminic agent or a structurally/functionally related compound, or a pharmaceutically acceptable salt, prodrug, solvate, hydrate or clathrate thereof.

4. A method of managing cancer, which comprises administering to a patient in need of such management a prophylactically effective amount of an antihistaminic agent or a structurally/functionally related compound, or a pharmaceutically acceptable salt, prodrug, solvate, hydrate or clathrate thereof.

5. The method of claim 3 or 4 wherein the antihistaminic agent or structurally/functionally related compound is perphenazine, sertralin, thioridazine, chlorpromazine, paroxetine, flupentixol, fluphenazine, hydroxyzine or promethazine.

6. The method of claim 3 or 4 wherein the cancer is leukemia, lymphoma, melanoma, or cancer of the liver, lung, pancreas, stomach, thyroid, larygopharnx, skin, uterus, breast, colon, cervix, ovary, testis, prostate or rectum.

7. The method of claim 6 wherein the cancer is leukemia, melanoma, or cancer of breast, colon or lung.

8. The method of claim 3 or 4 wherein the cancer is primary or metastatic.

9. A method of treating cancer, which comprises administering to a patient in need of such treatment an antihistaminic agent or a structurally/functionally related compound, or a pharmaceutically acceptable salt, prodrug, solvate, hydrate or clathrate thereof, and a second anti-cancer agent, or a pharmaceutically acceptable salt, prodrug, solvate, hydrate or clathrate thereof.

10. A method of managing cancer, which comprises administering to a patient in need of such management a prophylactically effective amount of an antihistaminic agent or a structurally/functionally related compound, or a pharmaceutically acceptable salt, prodrug, solvate, hydrate or clathrate thereof, and a second anti-cancer agent, or a pharmaceutically acceptable salt, prodrug, solvate, hydrate or clathrate thereof.

11. The method of claim 9 or 10 wherein the antihistaminic agent or structurally/functionally related compound is perphenazine, sertralin, thioridazine, chlorpromazine, paroxetine, flupentixol, fluphenazine, hydroxyzine or promethazine.

12. The method of claim 9 or 10 wherein the cancer is leukemia, lymphoma, melanoma, or cancer of the liver, lung, pancreas, stomach, thyroid, larygopharnx, skin, uterus, breast, colon, cervix, ovary, testis, prostate or rectum.

13. The method of claim 12 wherein the cancer is leukemia.

14. The method of claim 13 wherein the second anti-cancer agent is prednisone, vincristine, anthracycline, asparaginase, cytarabine, etoposide, cyclophosphamide, methotrexate, leucovorin, cytosine arabinose, corticosteroids, daunorubicin, idarubicin, 6-thioguanine, chlorambucil, fludarabine, interferon α, deoxyconformycin, 2-chlorodeoxyadenosine, hydroxyurea, 6-myelopurine, 6-thioguanine or melpharan.

15. The method of claim 12 wherein the cancer is melanoma.

16. The method of claim 15 wherein the second anti-cancer agent is dacarbazine, nitrosoureas carmustine, lomustine, cisplatin or interleukin-2.

17. The method of claim 12 wherein the cancer is breast cancer.

18. The method of claim 17 wherein the second anti-cancer agent is cyclophosphamide, 5-fluorouracil, methotrexate, doxorubicin, tamoxifen, progestins, aromatase inhibitors, aminoglutethimide, letrozole, paclitaxel, docetaxel, navelbine, capecitabine, mitomycin C, prednisone, taxane or vinblastine.

19. The method of claim 12 wherein the cancer is colon cancer.

20. The method of claim 19 wherein the second anti-cancer agent is fluorouracil, folinic acid, levamisole, leucovorin, oxaliplatin or irinotecan.

21. The method of claim 12 wherein the cancer is lung cancer.

22. The method of claim 21 wherein the second anti-cancer agent is cisplastin, topoisomerase inhibitors, carboplatin, vinorelbine, vincristine, vinblastine, docetaxel, oxaliplatin or paclitaxel.

23. A pharmaceutical composition comprising an antihistaminic agent or a structurally/functionally related compound, or a pharmaceutically acceptable salt, prodrug, solvate, hydrate or clathrate thereof, and a second anti-cancer agent, or a pharmaceutically acceptable salt, prodrug, solvate, hydrate or clathrate thereof.

24. The pharmaceutical composition of claim 23, wherein the antihistaminic agent or structurally/functionally related compound is perphenazine, sertralin, thioridazine, chlorpromazine, paroxetine, flupentixol, fluphenazine, hydroxyzine or promethazine.

25. The pharmaceutical composition of claim 23, wherein the second anti-cancer agent is prednisone, vincristine, anthracycline, asparaginase, cytarabine, etoposide, cyclophosphamide, methotrexate, leucovorin, cytosine arabinose, corticosteroids, daunorubicin, idarubicin, 6-thioguanine, chlorambucil, fludarabine, interferon α, deoxyconformycin, 2-chlorodeoxyadenosine, hydroxyurea, 6-myelopurine, melphalan, dacarbazine, nitrosoureas carmustine, lomustine, cisplatin, interleukin-2, 5-fluorouracil, doxorubicin, tamoxifen, progestins, aromatase inhibitors, aminoglutethimide, letrozole, paclitaxel, docetaxel, navelbine, capecitabine, mitomycin C, taxane, vinblastine, folinic acid, levamisole, irinotecan, topoisomerase inhibitors, carboplatin or vinorelbine.

26. A single unit pharmaceutical dosage form comprising an antihistaminic agent or a structurally/functionally related compound, or a pharmaceutically acceptable salt, prodrug, solvate, hydrate or clathrate thereof, and a second anti-cancer agent, or a pharmaceutically acceptable salt, prodrug, solvate, hydrate or clathrate thereof.

27. A method of determining whether a cancer patient will respond to a cancer treatment comprising testing for overexpression of TPT1 gene in cancer cells from the patient, wherein the cancer treatment is administration of an antihistaminic agent or a structurally/functionally related compound, or a pharmaceutically acceptable salt, prodrug, solvate, hydrate or clathrate thereof.

28. The method of claim 27, wherein the antihistaminic agent or structurally/functionally related compound is perphenazine, sertralin, thioridazine, chlorpromazine, paroxetine, flupentixol, fluphenazine, Atranine A or Atranine C.

29. The method of claim 27, wherein the overexpression results in about 20% or more TCTP formation as compared with normal cells.

30. The method of claim 29, wherein the overexpression results in about 100% or more TCTP formation as compared with normal cells.

31. The method of claim 30, wherein the overexpression results in about 200% or more TCTP formation as compared with normal cells.

32. A method of determining effectiveness of a cancer treatment comprising: obtaining cancer cells from a patient being treated at a first time and at a second time; and determining if expression of TPT1 gene in cells obtained at the second time is less than the expression of TPT1 gene in cells obtained at the first time; wherein the first time is earlier than the second time.

33. The method of claim 32, wherein the cancer treatment is administration of an antihistaminic agent or a structurally/functionally related compound, or a pharmaceutically acceptable salt, prodrug, solvate, hydrate or clathrate thereof.

34. The method of claim 33, wherein the antihistaminic agent or structurally/functionally related compound is perphenazine, sertralin, thioridazine, chlorpromazine, paroxetine, flupentixol, fluphenazine, Atranine A or Atranine C.
